(19) United States
(12) Patent Application Publication
(10) Pub. No.: US 2021/0077575 A1
(43) Pub. Date: Mar. 18, 2021

(54) NANOPARTICLES FOR THE TARGETED DELIVERY OF THERAPEUTIC POLYPEPTIDES

(71) Applicant: Cedars-Sinai Medical Center, Los Angeles, CA (US)

(72) Inventor: Lali K. MEDINA-KAUWE, Porter Ranch, CA (US)

(21) Appl. No.: 16/959,648

(22) PCT Filed: Dec. 28, 2018
(86) PCT No.: PCT/US18/67998

§ 371 Date: Jul. 1, 2020

Related U.S. Application Data
(60) Provisional application No. 62/612,812, filed on Jan. 2, 2018.

(51) Int. Cl. A61K 38/16; A61K 47/64; A61K 47/69; A61K 39/395; C07K 16/32; A61K 31/517; A61P 35/00

(52) U.S. Cl. CPC A61K 38/168; A61K 47/645; A61K 47/6931; A61K 2039/505; C07K 16/32; A61K 31/517; A61P 35/00; A61K 39/39558

(57) ABSTRACT

Nanoparticles can be useful for delivering therapeutic agents, such as anticancer agents to diseased cells. The nanoparticles include a carrier polypeptide and a cargo, which can be bind through electrostatic interactions to form a nanoparticle composition. An exemplary composition comprises nanoparticles comprising a carrier polypeptide comprising a penton base segment and a binding segment: and a polypeptide cargo comprising a tag segment that binds to the binding segment of the carrier poly peptide through an electrostatic interaction. An exemplary carrier polypeptide comprises a person base segment and a negatively-charged binding segment, which can bind to a positively charged cargo. The carrier polypeptide can also include a cell-targeting segment which can target the nanoparticle to a cell. Compositions comprising nanoparticles can be administered to a subject for the treatment of disease, such as cancer.

### CROSS-REFERENCE TO RELATED APPLICATIONS

This application claims priority benefit of U.S. Provisional Application No. 62/612,812, filed Jan. 2, 2018, the disclosure of which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761542001040_SEQLIST.TXT, date recorded: Dec. 28, 2018, size: 39 KB).

### FIELD OF THE INVENTION

The present invention relates to carrier polypeptides, nanoparticle compositions, and methods of treating disease, such as cancer, using such nanoparticle compositions.

### BACKGROUND

Effective delivery of chemotherapeutic agents to cancer cells is often limited by ineffective penetration of the cellular membrane. Certain cellular processes, such as endocytosis, can be exploited to penetrate the outer cellular membrane of the cancer cells, but delivery of the chemotherapeutic agent is further limited by endosomal escape. Recent development of a carrier polypeptide. HerPBK10, has been shown to be effective in delivering nucleic acid and corrole cargos to cancer cells. See, for example, U.S. Pat. No. 9,078,927; US 2012/004181; WO 2014/022811; and WO 2014/182868. The cargo binds to the decalysine (K10) segment of the HerPBK10 construct, resulting in the assembly of nanoparticles. The Her segment of the HerPBK10 carrier polypeptide can then target the nanoparticles to certain cancer cells.

Certain cytotoxic polypeptides, such as ricin or diphtheria toxin, have long been considered as potential chemotherapeutic agents. Although these compounds are effective at killing cells, development into useful chemotherapeutics has been limited due to the risk of off-target delivery.

The disclosures of all publications, patents, and patent applications referred to herein are each hereby incorporated herein by reference in their entireties. To the extent that any reference incorporated by references conflicts with the instant disclosure, the instant disclosure shall control.

### SUMMARY OF THE INVENTION

In one aspect, there is provided a composition comprising nanoparticles comprising a carrier polypeptide comprising a penton base segment and a binding segment, and a polypeptide cargo comprising a tag segment that binds to the binding segment of the carrier polypeptide through an electrostatic interaction.

In some embodiments, the tag segment is heterologous to the rest of the polypeptide cargo. In some embodiments, the tag segment is autologous to the rest of the polypeptide cargo. In some embodiments, the tag segment is at the C-terminus or the N-terminus of the polypeptide cargo. In some embodiments, the tag segment is cleavable. In some embodiments, the tag segment is internal to the polypeptide cargo. In some embodiments, the tag segment is about 4 amino acids to about 20 amino acids in length.

In some embodiments, the binding segment is positively charged. In some embodiments, the binding segment comprises poly-lysine or poly-arginine. In some embodiments, the binding segment comprises decalysine. In some embodiments, the tag segment is negatively charged. In some embodiments, the tag segment comprises poly-aspartic acid or poly-glutamic acid. In some embodiments, the tag segment comprises deca-aspartic acid.

In some embodiments, the binding segment is negatively charged. In some embodiments, the binding segment comprises poly-aspartic acid or poly-glutamic acid. In some embodiments, the binding segment comprises deca-aspartic acid. In some embodiments, the tag segment is positively charged. In some embodiments, the tag segment comprises poly-lysine or polyarginine. In some embodiments, the tag segment comprises deca-lysine.

In some embodiments, the polypeptide cargo comprises a therapeutic polypeptide. In some embodiments, the polypeptide cargo comprises a cytotoxic polypeptide. In some embodiments, the cytotoxic polypeptide is a protein-synthesis inhibitor. In some embodiments, the protein-synthesis inhibitor is gelonin or a variant thereof.

In some embodiments, the polypeptide cargo is about 5 kDa to about 50 kDa. In some embodiments, the polypeptide cargo is less than about 5 kDa.

In some embodiments, the molar ratio of the carrier polypeptide to the polypeptide cargo is about 3:1 to about 8:1.

In some embodiments, the carrier polypeptide further comprises a cell-targeting segment. In some embodiments, the cell-targeting segment binds a mammalian cell. In some embodiments, the cell-targeting segment binds a diseased cell. In some embodiments, the cell-targeting segment binds a cancer cell. In some embodiments, the cancer cell is a HER3+ cancer cell or a c-MET+ cancer cell. In some embodiments, the cell-targeting segment binds a target molecule on the surface of a cell. In some embodiments, the target molecule is a receptor. In some embodiments, the receptor HER3 or c-MET. In some embodiments, the cell-targeting segment comprises a ligand that specifically binds the receptor. In some embodiments, the cell-targeting segment comprises (i) Heregulin or a variant thereof; (ii) Internalin B or a variant thereof; or (iii) hepatocyte growth factor or a variant thereof.

In some embodiments, the penton base segment comprises an amino acid sequence according to SEQ ID NO: 1. In some embodiments, the penton base segment comprises a penton base variant.

In another aspect, there is provided a carrier polypeptide comprising a penton base segment and a negatively-charged binding segment. In some embodiments, the negatively-charged binding segment comprises poly-aspartic acid. In some embodiments, the negatively-charged binding segment comprises deca-aspartic acid. In some embodiments, the carrier polypeptide further comprises a cell-targeting segment. In some embodiments, the cell-targeting segment binds a mammalian cell. In some embodiments, the cell-targeting segment binds a diseased cell. In some embodiments, the cell-targeting segment binds a cancer cell. In some embodiments, the cancer cell is a HER3+ cancer cell or a c-MET+ cancer cell. In some embodiments, the cell-targeting segment binds a target molecule on the surface of a cell. In some embodiments, the target molecule is a receptor. In some embodiments, the receptor HER3 or c-MET. In some embodiments, the cell-targeting segment comprises a ligand that specifically binds the receptor. In some embodiments, the cell-targeting segment comprises (i) Heregulin or a variant thereof; (ii) Internalin B or a variant thereof; or (iii) hepatocyte growth factor or a variant thereof. In some embodiments, the penton base segment comprises an amino acid sequence according to SEQ ID NO: 1. In some embodiments, the penton base segment comprises a penton base variant.

In another aspect, there is provided a composition comprising nanoparticles comprising the carrier polypeptide described above; and a positively-charged cargo bound to the negatively-charged binding segment of the carrier polypeptide through an electrostatic interaction. In some embodiments, the cargo is a polypeptide cargo. In some embodiments, the cargo comprises a therapeutic agent. In some embodiments, the cargo comprises a cytotoxic agent.

In some embodiments of the composition described above, the average size of the nanoparticles in the composition is about 100 nm or less. In some embodiments, the nanoparticles in the composition have a polydispersity index of about 0.3 or less.

In another aspect, there is provided a pharmaceutical composition comprising the composition described above and a pharmaceutically acceptable excipient.

In another aspect, there is provided a method of treating a disease in a subject comprising administering an effective amount of the composition described above to the subject. In some embodiments, the disease is cancer. In some embodiments, the cancer is a HER3+ cancer and the carrier polypeptide comprises a cell-targeting segment that binds to HER3; or the cancer is a c-MET+ cancer and the carrier polypeptide comprises a cell-targeting segment that binds to c-MET. In some embodiments, the method further comprises administering an additional therapy to the subject. In some embodiments, the additional therapy is administered prior to administering the composition comprising the nanoparticles. In some embodiments, the additional therapy is administered after administering the composition comprising the nanoparticles. In some embodiments, the additional therapy is administered contemporaneous to administering the composition comprising the nanoparticles. In some embodiments, the additional therapy comprises administering a HER2 antibody to the subject. In some embodiments, the HER2 antibody is trastuzumab, pertuzumab, or a combination thereof. In some embodiments, the additional therapy comprises administering a HER2 inhibitor. In some embodiments, the HER2 inhibitor is lapatinib.

In another aspect, there is provided a method of making the nanoparticle composition described above comprising combining the carrier polypeptide and the cargo.

In another aspect, there is provided a method of delivering a polypeptide cargo to a cell comprising contacting the cell with the composition comprising the nanoparticles.

### BRIEF DESCRIPTION OF THE DRAWING

FIG. 1A illustrates a schematic of one example of a carrier polypeptide comprising a cell-targeting segment (namely, Her), a penton base segment, and a binding segment (namely K10) that can bind to a cargo through electrostatic interactions.

FIG. 1B illustrates a schematic of one example of a carrier polypeptide comprising a penton base segment and a negatively-charged binding segment (namely D10).

FIG. 1C illustrates a schematic of one example of a carrier polypeptide bound to a polypeptide cargo through electrostatic interactions.

FIGS. 2A-F presents particle size distribution as determined by dynamic light scattering (distribution by number). FIG. 2C shows particle size after combining an exemplary HerPBK10 carrier polypeptide with GFPD10. The HerPBK10 alone (FIG. 2B) and GFPD10 alone (FIG. 2A) are shown as a comparison. FIG. 2F shows particle size after combining an exemplary HerPBK10 carrier polypeptide with GeloninD10. The HerPBK10 (FIG. 2E) and GeloninD10 (FIG. 2D) are shown as a comparison.

FIG. 3A presents particle size distribution by number as determined by dynamic light scattering for HerPBK10-GFPD10 nanoparticles. FIG. 3B presents particle size distribution by number as determined by dynamic light scattering for HerPBK10-GeloninD10 nanoparticles.

FIGS. 4A-E shows confocal microscopy images of a time course intracellular trafficking assay. HER3+ cancer cells (SK-MEL-2 metastatic melanoma cells) were treated with HerPBK10-GFPD10 nanoparticles. The cells were fixed and immunocytofluoresence performed using an anti-AD5 antibody (which recognizes the penton base (PB) segment) to visualize HerPBK10 and an anti-GFP antibody to visualize GFPD10. The cells were counterstained using DAPI to stain the nucleus and rhodamine phalloidin to stain actin. Images were captured after 0 minutes (FIG. 4A), 15 minutes (FIG. 4B), 30 minutes (FIG. 4C), 60 minutes (FIG. 4D), and 120 minutes (FIG. 4E).

FIG. 5 shows the biodistribution of green fluorescent protein with a deca-aspartic acid tag (“GFPD10”), a fusion polypeptide containing a Her segment and green fluorescent protein (“Her-GFP”) or a nanoparticle containing HerPBK10 and GFPD10 (“HerPBK10-GFPD10). Biodistribution is shown in the subcutaneous tumors, lung, heart, liver, spleen, and kidneys of a mouse receiving the tested composition.

FIG. 6 shows the results of a time course assay of intracellular trafficking of HerPBK10 or HerPBK10-GFPD10 nanoparticles to A375-MA2 cells using an antibody that binds the penton base segment (“PB”) of HerPBK10.

FIG. 7 shows the results of a cell survival assay from MDA-MB-435 cells treated with gelonin having a C-terminal deca-aspartic acid tag (GeloninD10) or nanoparticles containing a HerPBK10 carrier polypeptide and Gelonin D10 (“HerPBK10-GeloninD10).

### DETAILED DESCRIPTION OF THE EMBODIMENTS

Provided herein there is a composition comprising nanoparticles, the nanoparticles comprising (i) a carrier polypeptide comprising a penton base segment and a binding segment; and the polypeptide cargo comprising a tag segment that binds to the binding segment of the carrier polypeptide through an electrostatic interaction. In some embodiments, the tag segment is heterologous to the rest of the polypeptide cargo. In some embodiments, the tag segment is at the C-terminus or the N-terminus of the polypeptide cargo, and in some embodiments the tag segment is internal to the polypeptide cargo. In some embodiments, the binding segment of the carrier polypeptide is positively charged, and the tag segment of the polypeptide cargo is negatively charged. In some embodiments, the binding segment of the carrier polypeptide is negatively charged, and the tag segment of the polypeptide cargo is positively charged.

Further provided herein there is a carrier polypeptide comprising a penton base segment and a negatively-charged binding segment. In some embodiments, there is a composition comprising the carrier polypeptide comprising the penton base segment and the negatively-charged binding segment; wherein the negatively-charged binding segment binds to a positively-charged cargo through an electrostatic interaction. In some embodiments, there is a composition comprising nanoparticle comprising a carrier polypeptide comprising a penton base segment and a negatively-charged binding segment that binds to a positively-charged cargo through an electrostatic interaction.

In some embodiments, the carrier polypeptide further comprises a cell-targeting segment. The cell-targeting segment can bind to a mammalian cell, such as a diseased cell (such as a cancer cell). For example, the cell-targeting segment can bind a target molecule on the surface of a cell (such as a receptor on the surface of the cell).

Also provided herein there is a method of treating a disease (such as cancer) in a subject comprising administering an effective amount of the composition according to the nanoparticle composition described herein.

Further provide there is a method of making the nanoparticle composition described herein.

Carrier polypeptides and cargo (for example, the polypeptide cargo or the positively charged cargo) can be combined to form nanoparticles. Without being bound by theory, it is believed that the cargo binds to the binding segment of the carrier polypeptide via electrostatic interactions, and is located within the core of the nanoparticle. In some embodiments, the cargo (such as a polypeptide cargo) includes a tag segment that binds to the binding segment of the carrier polypeptide. It is further believed, without being bound by theory, that the penton base segment oligomerizes to form a shell around the cargo. The assembled nanoparticle can then be useful for transporting and delivering the cargo, for example to a target cell. For example, in some embodiments, a cell-targeting segment is presented by the nanoparticle, and the cell-targeting segment can bind to a target molecule on the surface of a targeted cell. The carrier polypeptide can bind to a cell, which can then internalize the nanoparticle (including the cargo) into an endosome. The penton base segment allows for endosomal escape of the carrier polypeptide and cargo into the cellular cytoplasm.

Surprisingly, the carrier polypeptides forms nanoparticles with polypeptide cargos, which can be significantly larger than other cargos described as binding to carrier polypeptides, such as corroles. See, for example, WO 2014/182868. For example, in some embodiments, the polypeptide cargo is about 5 kDa or more. Additionally, it is surprising that the electrostatic interactions between the binding segment of the carrier polypeptide and the tag segment of the polypeptide cargo is sufficiently strong to allow stable nanoparticle formation, particularly when the tag segment of the polypeptide cargo can be a relatively small segment compared to the rest of the polypeptide cargo. For example, as shown in the Examples, a deca-aspartic acid tag segment (D10) fused to a green fluorescent protein (about 33 kDa) bound to a deca-lysine binding segment of a carrier polypeptide with sufficient strength to form a stable nanoparticle composition.

The carrier polypeptide can optionally comprise a cell-targeting segment, which can direct the nanoparticles to a particular type of cell. For example, in some embodiments, the carrier polypeptide comprises a Heregulin sequence or variant thereof, which can target HER3+ cells. In some embodiments, the carrier polypeptide comprises an Internalin B sequence or variant thereof, or hepatocyte growth factor or a variant thereof, either of which can target c-MET+ cells. The carrier polypeptide need not include a cell-targeting segment. For example, in some embodiments, the penton base binds integrin through an RGD (arginine-glycine-aspartic acid) motif present in the penton base, and can be used to deliver the cargo to cells with integrin on their surface (such as cells that are upregulated for integrin expression).

As used herein, the singular forms “a,” “an,” and “the” include the plural reference unless the context clearly dictates otherwise.

Reference to “about” a value or parameter herein includes (and describes) variations that are directed to that value or parameter per se. For example, description referring to “about X” includes description of “X”.

A “bioactive segment” of a polypeptide cargo is any portion of a polypeptide cargo that exhibits bioactivity. The bioactive segment need not exclude a binding segment of a polypeptide cargo. Bioactivity refers to any activity that alters the biological function of a cell, including, but not limited to, cytotoxic activity, enzymatic activity, or inhibition of protein binding.

“D10” is used herein to refer to deca-aspartic acid (i.e., ten contiguous aspartic acid amino acid residues).

The term “effective” or “effective amount” is used herein, unless otherwise indicated, to describe an amount of a compound or component which, when used within the context of its use, produces or effects an intended result, whether that result relates to the treatment of an infection or disease state or as otherwise described herein.

An “electrostatic interaction” refers to a non-covalent attractive force between two or more atoms based on an ionic interaction, a hydrogen bonding interaction, or a dipole-dipole interaction.

“Her” is used herein to refer to a Heregulin polypeptide.

“InlB” is used herein to refer to an Internalin B polypeptide.

“K10” is used herein to refer to deca-lysine (i.e., ten contiguous lysine amino acid residues).

“PB” is used herein to refer to a penton base segment.

A “polypeptide cargo” refers to any peptide with two or more peptide bonds that can be carried by a carrier polypeptide.

The term “pharmaceutically acceptable” when used to refer to a compound or composition means that the compound or composition is suitable for administration to a subject, including a human subject, to achieve the treatments described herein, without unduly deleterious side effects in light of the severity of the disease and necessity of the treatment.

The term “subject” or “patient” is used synonymously herein to describe a mammal. Examples of a subject include a human or animal (including, but not limited to, dog, cat, rodent (such as mouse, rat, or hamster), horse, sheep, cow, pig, goat, donkey, rabbit, or primates (such as monkey, chimpanzee, orangutan, baboon, or macaque)).

The terms “treat,” “treating,” and “treatment” are used synonymously herein to refer to any action providing a benefit to a subject afflicted with a disease state or condition, including improvement in the condition through lessening, inhibition, suppression, or elimination of at least one symptom, delay in progression of the disease, delay in recurrence of the disease, or inhibition of the disease.

A cell that exhibits “upregulated expression” or “overexpression” for a particular protein (e.g., HER3+ or c-MET+) is said to be upregulated when the cell presents more of that protein relative to a cell that is not upregulated for that protein.

It is understood that aspects and variations of the invention described herein include “consisting” and/or “consisting essentially of” aspects and variations.

It is to be understood that one, some or all of the properties of the various embodiments described herein may be combined to form other embodiments of the present invention.

The section headings used herein are for organizational purposes only and are not to be construed as limiting the subject matter described.

### Carrier Polypeptides

The carrier polypeptide includes a penton base segment and a binding segment. The binding segment can bind a cargo, for example a charged polypeptide cargo described in further detail herein, through an electrostatic interaction. For example, in some embodiments, the binding segment of the carrier polypeptide is positively charged and the cargo, such as a polypeptide cargo, includes a negatively-charged tag segment. In some embodiments, a positively-charged binding segment of the carrier polypeptide binds the negatively-charged tag segment of the polypeptide cargo. In some embodiments, the carrier polypeptide further includes a cell-targeting segment. Optionally, the carrier polypeptide further includes a linker (such as a poly-glycine) domain positioned between the binding segment and the penton base segment or between the cell-targeting segment (if present) and the penton base segment.

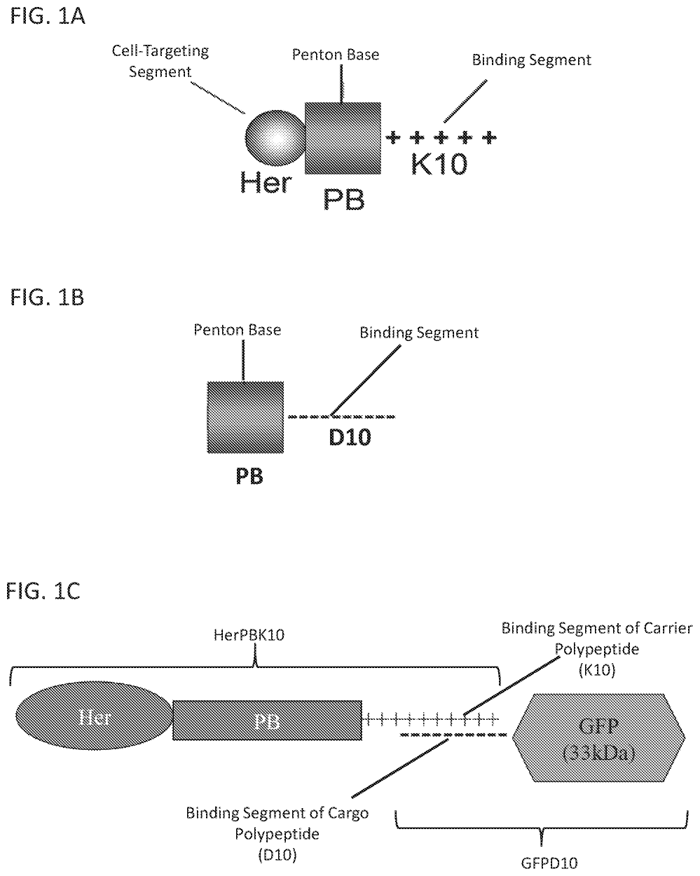

FIG. 1A illustrates one embodiment of a carrier polypeptide. In the illustrated example, the carrier polypeptide includes a cell-targeting segment (Heregulin in the illustrated example), a penton base segment, and a binding segment (K10 in the illustrated example). The cell-targeting segment is located towards the N-terminus relative to the penton base segment, and the binding segment is located towards the C-terminus relative to the penton base segment. FIG. 1B illustrates another embodiment of a carrier polypeptide comprising a penton base segment and a negatively-charged binding segment (D10 in the illustrated example). Although not illustrated in FIG. 1B, the carrier polypeptide optionally includes a cell-targeting segment.

The carrier polypeptide can be a recombinant fusion protein expressed and purified using known techniques. Although not illustrated in FIG. 1A or FIG. 1B, the carrier polypeptide can optionally include a linker between the cell-targeting segment and the penton base segment, or between the penton base segment and the binding segment. Also optionally, the carrier polypeptide can include an N-terminal tail or a C-terminal tail. For example, the carrier polypeptide can include an N-terminal or C-terminal tail which can be used to isolate the carrier polypeptide, which is optionally cleavable.

The penton base segment of the carrier polypeptide is a penton base protein or a variant thereof. The penton base protein is a major component of a viral capsid (such as an adenovirus capsid) that can be incorporated into a carrier polypeptide to form nanoparticles. See, for example, International Published Patent Application WO 2002/094318; U.S. Pat. No. 8,765,666; U.S. Published Application No. US 2015/240231; and U.S. Published Application No. US 2012/004181. By way of example, in some embodiments, the penton base segment is the adenovirus serotype 5 (Ad5) penton base protein or a variant thereof. SEQ ID NO: 1 is the wild-type amino sequence of the penton base segment from adenovirus serotype 5 (Ad5). In some embodiments, the penton base segment is about 80% identical or more (such as about 85% identical or more, about 90% identical or more, about 92% identical or more, about 95% identical or more, about 98% identical or more, or 99% identical or more) to SEQ ID NO: 1.

In some embodiments, the penton base segment is a penton base protein with one or more point mutations or truncations. Various mutations of the penton base segment are detailed in WO 2014/022811. In some embodiments, the penton base segment includes one or more point mutations or truncations that enhance localization of the carrier polypeptide (and thus, enhance localization of cargo bound to the carrier polypeptide) to the cytoplasm or nucleus of a cell. For example, in some embodiments, the penton base segment comprises one or more of a Met1Thr, Leu60Trp, Lys375Glu, Val449Met, or Pro469Ser point mutations. Amino acid numbering is made in reference to the wild-type penton base polypeptide of SEQ ID NO: 1.

Wild-type penton base segment includes an RGD (arginine-glycine-aspartic acid) motif. The RGD motif binds to cellular integrin. Thus, in some embodiments, the penton base segment targets integrin on the surface of cells. Use of the RGD motif in the penton base segment can be particularly useful when targeting cells with upregulate integrin expression, such as certain cancer cells with upregulated integrin expression. Nevertheless, in some embodiments, the RGD motif of the penton base segment is mutated (for example, to an EGD (glutamic acid-glycine-aspartic acid) motif).

The binding segment of the carrier polypeptide is able to bind the cargo of the nanoparticle, generally through an electrostatic interaction. In some embodiments, the binding segment is a heterologous segment (relative to the penton base segment) or a synthetic segment. In some embodiments, the binding segment of the carrier polypeptide is positively charged. For example, the binding segment of the carrier polypeptide can comprise a poly-lysine or poly-arginine motif. In some embodiments, the binding segment of the carrier polypeptide is a decalysine motif (that is, ten sequential lysine amino acids, or “K10,” as shown in SEQ ID NO: 2). In some embodiments, the binding segment of the carrier polypeptide is negatively charged. For example, the binding segment of the carrier polypeptide can comprise a poly-aspartic acid motif or a poly-glutamic acid motif. In some embodiments, the binding segment of the carrier polypeptide is a deca-aspartic acid motif (that is, ten sequential aspartic acid amino acids, or “D10” as shown in SEQ ID NO: 3.

The cell-targeting segment of the carrier polypeptide (if present) can bind to a target molecule present on the surface of a cell. Binding of the molecule by the cell-targeting segment allows the nanoparticle to be targeted to the cell. Thus, the targeted molecule present on the cell can depend on the targeted cell. In some embodiments, the targeted molecule is an antigen, such as a cancer antigen. In some embodiments, the cancer cell exhibits upregulated expression (i.e., overexpression) of the target molecule. The upregulated expression may be for example, an increase of about 10% or more, about 20% or more, about 30% or more, about 40% or more, about 50% or more, about 60% or more, about 70% or more, about 80% or more, about 90% or more, or about 100% or more. In some embodiments, the targeted molecule is a cell surface receptor, such as HER3 or c-MET. In some embodiments, the cell-targeting segment binds to 4-IBB, 5T4, adenocarcinoma antigen, alpha-fetoprotein, BAFF, C242 antigen, CA-125, carbonic anhydrase 9 (CA-IX), c-MET, CCR4, CD152, CD19, CD20, CD200, CD22, CD221, CD23 (IgE receptor), CD28, CD30 (TNFRSF8), CD33, CD4, CD40, CD44v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, hepatocyte growth factor (HGF), human scatter factor receptor kinase, IGF-1 receptor, IGF−1, IgG, L-CAM. IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, vimentin, Internalin B, bacterial invasin (Inv) protein, or a fragment thereof.

In some embodiments, the cell-targeting segment comprises an antibody, an antibody fragment (such as a Fab fragment, a F(ab′)2 fragment, a Fab′ fragment, or a single-chain variable (scFv) fragment), a cytokine, or a receptor ligand.

In some embodiment, the cell-targeting segment comprises a ligand that specifically binds to a receptor expressed on the surface of a cell. Exemplary ligands include a Heregulin sequence (or a variant thereof), an Internalin B sequence (or a variant thereof), or a hepatocyte growth factor sequence (or a variant thereof). The ligand variants retain specific binding for the targeted molecule. The variant can be, for example, a truncation variant, a point mutation variant, an insertion variant, or a deletion variant. Heregulin (which can be referred to as “Her”) can specifically bind to HER3. SEQ ID NO: 4 is an exemplary wild-type Her sequence. The heregulin segment generally includes the EGF-like domain from heregulin. In some embodiments, the heregulin segment includes the EGF-like domain and the Ig domain of heregulin. Internalin B can specifically bind to c-MET, and can also be referred to as “InlB”. SEQ ID NO: 5 is an exemplary wild-type InlB sequence. The variant can be, for example, any portion of the protein (e.g., Heregulin. Internalin B, or hepatocyte growth factor) that maintains specific binding activity to the target molecule, and can include a truncation, one or more point mutations, one or more amino acid insertions, or one or more amino acid deletions.

In some embodiments, the cell targeted by the cell-targeting segment is a mammalian cell, such as a human cell. In some embodiments, the cell is a diseased cell, such as a cancer cell. In some embodiment, the cell is a HER3+ cancer cell or a c-MET+ cancer cell. In some embodiment, the cell is a head and neck cancer cell, a pancreatic cancer cell, a breast cancer cell, a glial cancer cell, an ovarian cancer cell, a cervical cancer cell, a gastric cancer cell, a skin cancer cell, a colon cancer cell, a rectal cancer cell, a lung cancer cell, a kidney cancer cell, or a thyroid cancer cell. The cell-targeting segment can bind a molecule present on the surface of the targeted cell, which targets the nanoparticle to the targeted cell.

In some embodiments, the carrier polypeptide comprises a penton base segment and a binding segment.

In some embodiments, the carrier polypeptide comprises a penton base segment and a positively-charged binding segment. In some embodiments, the carrier polypeptide comprises a penton base segment and a poly-lysine segment. In some embodiments, the carrier polypeptide comprises a penton base segment and a deca-lysine segment. In some embodiments, the carrier polypeptide comprises a penton base segment and a poly-arginine segment.

In some embodiments, the carrier polypeptide comprises a penton base segment and a negatively-charged binding segment. In some embodiments, the carrier polypeptide comprises a penton base segment and a poly-aspartic acid segment. In some embodiments, the carrier polypeptide comprises a penton base segment and a deca-aspartic acid segment. In some embodiments, the carrier polypeptide comprises a penton base segment and a poly-glutamic acid segment.

In some embodiments, the carrier polypeptide comprises a cell-targeting segment, a penton base segment, and a binding segment. In some embodiments, the cell-targeting segment targets HER3 or c-MET.

In some embodiments, the carrier polypeptide comprises a cell-targeting segment, a penton base segment, and a positively-charged binding segment. In some embodiments, the carrier polypeptide comprises a cell-targeting segment, a penton base segment, and a poly-lysine segment. In some embodiments, the carrier polypeptide comprises a cell-targeting segment, a penton base segment, and a deca-lysine segment. In some embodiments, the carrier polypeptide comprises a cell-targeting segment, a penton base segment, and a poly-arginine segment. In some embodiments, the cell-targeting segment targets HER3 or c-MET.

In some embodiments, the carrier polypeptide comprises a cell-targeting segment, a penton base segment, and a negatively-charged binding segment. In some embodiments, the carrier polypeptide comprises a cell-targeting segment, a penton base segment, and a poly-aspartic acid segment. In some embodiments, the carrier polypeptide comprises a cell-targeting segment, a penton base segment, and a deca-aspartic acid segment. In some embodiments, the carrier polypeptide comprises a cell-targeting segment, a penton base segment, and a poly-glutamic acid segment. In some embodiments, the cell-targeting segment targets HER3 or c-MET.

In some embodiments, the carrier polypeptide comprises Heregulin or a variant thereof, a penton base segment, and a poly-lysine binding segment. In some embodiments, the carrier polypeptide comprises the amino acid sequence according to SEQ ID NO: 6. In some embodiments, the carrier polypeptide is HerPBK10 (SEQ ID NO: 6).

In some embodiments, the carrier polypeptide comprises Heregulin or a variant thereof, a penton base segment, and a poly-aspartic acid binding segment. In some embodiments, the carrier polypeptide comprises the amino acid sequence according to SEQ ID NO: 7. In some embodiments, the carrier polypeptide is HerPBD10 (SEQ ID NO: 7).

In some embodiments, the carrier polypeptide comprises Internalin B or a variant thereof, a penton base segment, and a poly-lysine binding segment. In some embodiments, the carrier comprises the amino acid sequence according to SEQ ID NO: 8. In some embodiments, the carrier polypeptide is InlBPBK10 (SEQ ID NO: 8).

In some embodiments, the carrier polypeptide comprises Internalin B or a variant thereof, a penton base segment, and a poly-aspartic acid binding segment. In some embodiments, the carrier comprises the amino acid sequence according to SEQ ID NO: 9. In some embodiments, the carrier polypeptide is InlBPBD10 (SEQ ID NO: 9).

In some embodiments, the carrier polypeptide comprises hepatocyte growth factor or a variant thereof, a penton base segment, and a poly-lysine binding segment.

In some embodiments, the carrier polypeptide comprises hepatocyte growth factor or a variant thereof, a penton base segment, and a poly-aspartic acid binding segment.

### Cargos

The cargo of the nanoparticle binds to the carrier polypeptide. For example, the cargo can bind to the binding segment of the carrier polypeptide through an electrostatic interaction. In some embodiments, the cargo (such as a polypeptide cargo) is positively charged, and the carrier polypeptide includes a negatively charged binding segment. In some embodiments, the cargo (such as a polypeptide cargo) comprises a tag segment that binds to the binding segment of the carrier polypeptide, for example through electrostatic interactions. For example, in some embodiments, the carrier polypeptide includes a positively-charged binding segment that binds to a negatively-charged tag of a polypeptide cargo through an electrostatic interaction. In some embodiments, the carrier polypeptide includes a negatively charged binding segment that binds to a positively-charged tag of the polypeptide cargo through an electrostatic interaction.

In some embodiments, the cargo that binds to the carrier polypeptide is positively charged, and the carrier polypeptide includes a negatively charged binding segment that binds to the positively charged cargo through an electrostatic interaction. For example, the positively charged cargo can have a surface with a net positively charge, and the positive surface charges mediate binding to the negatively charged binding segment of the carrier polypeptide. In some embodiments, the positively charged cargo is a polypeptide cargo. In some embodiments, the polypeptide cargo is a therapeutic agent, such as cytotoxic agent.

In some embodiments, the polypeptide cargo comprises a bioactive segment and a tag segment. In some embodiments, the polypeptide cargo comprises a fluorescent segment and a tag segment. The tag segment and the bioactive segment (or fluorescent segment) of the polypeptide cargo need not be distinct, and the tag segment can be part of or distinct from the bioactive segment (or fluorescent segment). For example, in some embodiments, the tag segment mediates electrostatic interactions between the polypeptide cargo and the binding segment of the carrier polypeptide, but also exhibits or contributes to bioactivity (or fluorescence).

The polypeptide cargo can be a natural polypeptide, a synthetic polypeptide, or a combination thereof. For example, in some embodiments the polypeptide cargo includes a natural polypeptide portion and a synthetic polypeptide portion. In some embodiments, the polypeptide cargo includes two natural polypeptide portions that are fused together (e.g., a heterologous fusion protein). In some embodiment the polypeptide cargo comprises a therapeutic polypeptide, for example a chemotherapeutic (i.e., cytotoxic) polypeptide. In some embodiments, the polypeptide cargo comprises a protein-synthesis inhibitor, such as gelonin or a variant thereof. Gelonin is a ribosome-inactivating protein of about 29 kDa in size, and has been shown to be effective against certain cancer cells. See, for example, Bai et al., Efficient Inhibition of Ovarian Cancer by Gelonin Toxin Gene Delivered by Biodegradable Cationic Heparin-Polyethyleneine Nanogels, International Journal of Medical Sciences, vol. 12 (5), pp. 397406 (May 8, 2015). Gelonin is an exemplary cytotoxic polypeptide, and other cytotoxic polypeptides may be used. Other cytotoxic polypeptides that can be used include ricin, saporin, and diphtheria toxin polypeptides. In some embodiments, the polypeptide cargo comprises a fluorescent polypeptide, such as green fluorescent protein.

In some embodiments, the polypeptide cargo is less than about 5 kDa (such as less than about 4.5 kDa, less than about 4 kDa, less than about 3.5 kDa, less than about 3 kDa, less than about 2.5 kDa, less than about 2 kDa, less than about 1.5 kDa, or less than about 1 kDa). In some embodiments, the polypeptide cargo is about 5 kDa or more (such as about 7.5 kDa or more, about 10 kDa or more, about 15 kDa or more, about 20 kDa or more, about 25 kDa or more, about 30 kDa or more, about 35 kDa or more, about 40 kDa or more, or about 45 kDa or more). In some embodiments, the polypeptide cargo is about 0.5 kDa to less than about 5 kDa (such as about 1 kDa to about 4 kDa, or about 2 kDa to about 3 kDa). In some embodiments, the polypeptide cargo is about 5 kDa to about 50 kDa (such as about 10 kDa to about 45 kDa, about 15 kDa to about 40 kDa, about 20 kDa, to about 35 kDa, or about 25 kDa to about 30 kDa).

In some embodiments, the polypeptide cargo is about 3 to about 500 amino acids in length (such as about 3 to about 10 amino acids, about 10 to about 20 amino acids, about 20 to about 50 amino acids, about 50 to about 100 amino acids, about 100 to about 200 amino acids, about 200 to about 350 amino acids, or about 350 to about 500 amino acids in length).

The polypeptide cargo can be any polypeptide with three or more amino acid residues that binds to the carrier polypeptide. The polypeptide cargo can include a tag segment, which includes one or more moieties (such as amino acid residues) that mediate the electrostatic interaction between the polypeptide cargo and the binding segment of the carrier polypeptide. Deletion or non-conservative derivation of the moieties in the tag segment of the polypeptide cargo can disrupt binding of the polypeptide cargo to the binding segment of the carrier polypeptide or prevent nanoparticle formation (although in some embodiments, deletion or non-conservative mutation of a portion of the moieties in the tag segment of the polypeptide cargo does not disrupt binding of the polypeptide cargo to the binding segment of the carrier polypeptide or prevent nanoparticle formation). In some embodiments, one or more of the moieties in the tag segment are amino acids, and deletion or non-conservative mutation of the amino acids in the tag segment of the polypeptide cargo can disrupt binding of the tag segment of the polypeptide cargo to the binding segment of the carrier polypeptide or prevent nanoparticle formation. The amino acids in the tag segment of the polypeptide cargo can be natural amino acids or synthetic amino acids.

In some embodiments, the tag segment of the polypeptide cargo is heterologous to the rest of the polypeptide cargo. For example, the tag segment can be a synthetic sequence attached to the rest of the polypeptide cargo (such as a recombinant fusion protein or fusion of polypeptides, such as after protein expression). In some embodiments, the tag segment of the polypeptide cargo is autologous to the rest of the polypeptide cargo. For example, the polypeptide cargo can include an autologous positively-charged region (i.e., the tag segment) and can bind to a negatively-charged binding segment of the carrier polypeptide. In some embodiments, the polypeptide cargo includes an autologous negatively-charged region and can bind to a positively-charged binding segment of the carrier polypeptide. In some embodiments, the tag segment of the polypeptide cargo is a contiguous amino acid sequence. In some embodiments, the tag segment of the polypeptide cargo is a non-contiguous amino acid sequence. For example, in some embodiments, the tag segment of the polypeptide cargo is a non-contiguous amino acid sequence that is clustered in the protein's tertiary structure.

In some embodiments, the tag segment of the polypeptide cargo is at the C-terminus or the N-terminus of the polypeptide cargo, which can be synthetically attached or as a portion of a natural polypeptide. In some embodiments, the tag segment of the polypeptide cargo is cleavable. For example, in some embodiment, a proteolytic cleavage site is included in the polypeptide cargo between a bioactive segment of the polypeptide cargo and the tag segment of the polypeptide cargo. In some embodiments, the tag segment of the polypeptide cargo is internal to the polypeptide cargo. That is, the tag segment need not be on the N-terminus or the C-terminus of the polypeptide cargo, but can be in any inner region of the polypeptide cargo, and can, for example, be part of a bioactive portion of the polypeptide cargo or separate two or more portion of the bioactive segment (or fluorescent segment) of the polypeptide cargo.

In some embodiments, the tag segment of the polypeptide cargo is one or more amino acids in length (such as 2 or more, 3 or more, 4 or more, 5 or more, 6 or more, 7 or more, 8 or more, 9 or more, or 10 or more amino acids in length). In some embodiments, the tag segment of the polypeptide cargo has a length of about 30 amino acids or less (such as about 20 or less, about 15 or less, about 10 or less, about 9 or less, about 8 or less, about 7 or less, about 6 or less, or about 5 or less). In some embodiments, the tag segment of the polypeptide cargo is about 3 amino acids in length to about 30 amino acids in length (such as about 4 amino acids to about 20 amino acids, about 5 amino acids to about 15 amino acids, or about 10 amino acids in length).

In some embodiments, the tag segment of the polypeptide cargo is positively charge. For example, in some embodiments, the binding segment of the carrier polypeptide is negatively charged, and the tag segment of the polypeptide cargo is positively charged, and the binding segment of the carrier polypeptide and the tag segment of the polypeptide cargo bind through an electrostatic interaction. In some embodiments, the tag segment of the polypeptide cargo comprises poly-lysine or poly-arginine, such as deca-lysine or deca-arginine. In some embodiments, the tag segment of the polypeptide cargo is negatively charge. For example, in some embodiments, the binding segment of the carrier polypeptide is positively charged, and the binding segment of the tag cargo is negatively charged, and the binding segment of the carrier polypeptide and the tag segment of the polypeptide cargo bind through an electrostatic interaction. In some embodiments, the tag segment of the polypeptide cargo comprises poly-aspartic acid or poly-glutamic acid, such as deca-aspartic acid or deca-glutamic acid.

### Nanoparticle Compositions

The nanoparticle compositions described herein include nanoparticles comprising a carrier polypeptide and a cargo. The carrier polypeptides are described in further detail herein, and include a penton base segment and a binding segment that binds to the polypeptide cargo. Optionally, the carrier polypeptide further includes a cell-targeting segment. The cargo can be a polypeptide cargo, as described in detail herein, or a positively charged cargo, also as described in detail herein.

The nanoparticles spontaneously form after combining the carrier polypeptide with the cargo. In some embodiments, the nanoparticle composition comprises carrier polypeptides and the cargo (such as a polypeptide cargo or a positively charged cargo) at a molar ratio of about 3:1 to about 8:1 (such as about 3:1 to about 3.5:1, about 3.5:1 to about 4:1, about 4:1 to about 4.5:1, about 4.5:1 to about 5:1, about 5:1 to about 5.5:1, about 5.5:1 to about 6:1, about 6:1 to about 6.5:1, about 6.5:1 to about 7:1, about 7:1 to about 7.5:1, about 7.5:1 to about 8:1, about 4:1, about 4.5:1, about 5:1, about 5.5:1, about 6:1, about 6.5:1, about 7:1, about 7.5:1, or about 8:1).

In some embodiments the nanoparticles in the nanoparticle composition have an average size of about 100 nm or less (such as about 90 nm or less, about 80 nm or less, about 70 nm or less, about 60 nm or less, about 50 nm or less, or about 40 nm or less). In some embodiments, nanoparticles have an average size between about 10 nm and about 100 nm (such as between about 10 nm and about 30 nm, between about 30 nm and about 50 nm, between about 50 nm and about 70 nm, or between 70 nm and about 100 nm.

In some embodiments, the nanoparticles have a polydispersity index of about 0.2 or lower (such as about 0.15 or lower, about 0.1 or lower, about 0.05 or lower, about 0.04 or lower, about 0.03 or lower, about 0.02 or lower, or about 0.01 or lower). In some embodiments, nanoparticles comprising a HerPBK10 carrier polypeptide and a Gelonin-D10 polypeptide cargo have a polydispersity index of about 0.2 or lower (such as about 0.15 or lower, about 0.1 or lower, about 0.05 or lower, or about 0.04 or lower). Polydispersity index (PDI) can be measured by dynamic light scattering (distribution by number) using the formula

$$P D I = {( \frac{σ}{d} )}^{2} ,$$

wherein d is the mean diameter of the nanoparticles and a is the standard deviation of the diameter of the nanoparticles. Nanoparticles with a polydispersity index less than 0.1 can be referred to as monodisperse nanoparticles.

The nanoparticle compositions described herein can be made by combining the carrier polypeptides with the cargo (such as the positively-charged cargo or the polypeptide cargo). In some embodiments, the carrier polypeptides and the cargo are incubated together to form the nanoparticles. In some embodiments, the carrier peptide and the cargo are combined at a molar ratio of a molar ratio of about 3:1 to about 8:1 (such as about 3:1 to about 3.5:1, about 3.5:1 to about 4:1, about 4:1 to about 4.5:1, about 4.5:1 to about 5:1, about 5:1 to about 5.5:1, about 5.5:1 to about 6:1, about 6:1 to about 6.5:1, about 6.5:1 to about 7:1, about 7:1 to about 7.5:1, about 7.5:1 to about 8:1, about 4:1, about 4.5:1, about 5:1, about 5.5:1, about 6:1, about 6.5:1, about 7:1, about 7.5:1, or about 8:1). In some embodiments, the carrier polypeptide and the cargo are incubated at about 4° C. to about 22° C. such as between about 4° C. and about 15° C., or between about 4° C. and about 10° C. In some embodiments, the carrier polypeptide and the cargo incubate for less than about 30 minutes, about 30 minutes or more, about 1 hour or more, or about 2 hours or more.

In some embodiments, carrier polypeptide or cargo that does not assemble into nanoparticles is removed from the composition comprising the nanoparticles. For example, in some embodiments, the nanoparticle composition is subjected to a purification step, such as size exclusion chromatography. In some embodiments, the unbound components are separated from the nanoparticles by ultracentrifugation. For example, in some embodiments, the composition is added to a centrifugal filter with a molecular weight cutoff of about 100 kD or less, about 80 kD or less, about 70 kD or less, about 60 kD or less, about 50 kD or less, about 40 kD or less, about 30 kD or less, or about 20 kD or less.

Optionally, the resulting nanoparticle composition is subjected to buffer exchange, for example by dialysis, ultracentrifugation, or tangential flow filtration. In some embodiments, the nanoparticles are concentrated, for example by ultracentrifugation.

The nanoparticle composition can undergo further processing steps. For example in some embodiments, the nanoparticle composition is sterilized, for example by sterile filtration. In some embodiments, the nanoparticle composition is dispensed into a vial (which may then be sealed). In some embodiments, the nanoparticle composition is lyophilized, thereby forming a dry nanoparticle composition. In some embodiments, the nanoparticle composition is formulated to form a pharmaceutical composition, for example by adding one or more pharmaceutically acceptable excipients.

In some embodiments, a composition comprises nanoparticles comprising a carrier polypeptide comprising a penton base segment and a binding segment; and a polypeptide cargo comprising a tag segment bound to the binding segment of the carrier polypeptide through an electrostatic interaction. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBK10. In some embodiments, the cell-targeting segment is Internalin B or a variant thereof. In some embodiments, the carrier polypeptide is InlBPBK10. In some embodiments the cell-targeting segment is hepatocyte growth factor or a variant thereof. In some embodiments, the tag segment is about 4 amino acids to about 20 amino acids in length. In some embodiments, the polypeptide cargo comprises a therapeutic polypeptide, such as a cytotoxic polypeptide. In some embodiments, the cytotoxic polypeptide comprises a protein-synthesis inhibitor, such as gelonin or a variant thereof. In some embodiments, the polypeptide cargo is about 5 kDa to about 50 kDa. In some embodiments, the polypeptide cargo is less than about 5 kDa. In some embodiments, the molar ratio of the carrier polypeptide to the polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, a composition comprises nanoparticles comprising a carrier polypeptide comprising a penton base segment and a binding segment; and a polypeptide cargo comprising a tag segment heterologous to the rest of the polypeptide cargo that binds to the binding segment of the carrier polypeptide through an electrostatic interaction. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBK10. In some embodiments, the cell-targeting segment is Internalin B or a variant thereof. In some embodiments, the carrier polypeptide is InlBPBK10. In some embodiments, the cell-targeting segment is hepatocyte growth factor (HGF) or a variant thereof. In some embodiments, the carrier polypeptide is HGFPBK10. In some embodiments, the heterologous tag segment is about 4 amino acids to about 20 amino acids in length. In some embodiments, the polypeptide cargo comprises a therapeutic polypeptide, such as a cytotoxic polypeptide. In some embodiments, the cytotoxic polypeptide comprises a protein-synthesis inhibitor, such as gelonin or a variant thereof. In some embodiments, the polypeptide cargo is about 5 kDa to about 50 kDa. In some embodiments, the polypeptide cargo is less than about 5 kDa. In some embodiments, the molar ratio of the carrier polypeptide to the polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, a composition comprises nanoparticles comprising a carrier polypeptide comprising a penton base segment and a binding segment; and a polypeptide cargo comprising a tag segment heterologous to the rest of the polypeptide cargo and at the N-terminus or the C-terminus of the polypeptide cargo, wherein the tag segment binds to the binding segment of the carrier polypeptide through an electrostatic interaction. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBK10. In some embodiments, the cell-targeting segment is Internalin B or a variant thereof. In some embodiments, the carrier poly peptide is InlBPBK10. In some embodiments, the cell-targeting segment is hepatocyte growth factor (HGF) or a variant thereof. In some embodiments, the carrier polypeptide is HGFPBK10. In some embodiments, the tag segment is about 4 amino acids to about 20 amino acids in length. In some embodiments, the polypeptide cargo comprises a therapeutic polypeptide, such as a cytotoxic polypeptide. In some embodiments, the cytotoxic polypeptide comprises a protein-synthesis inhibitor, such as gelonin or a variant thereof. In some embodiments, the polypeptide cargo is about 5 kDa to about 50 kDa. In some embodiments, the polypeptide cargo is less than about 5 kDa. In some embodiments, the molar ratio of the carrier polypeptide to the polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, a composition comprises nanoparticles comprising a carrier polypeptide comprising a penton base segment and a positively-charged binding segment; and a polypeptide cargo comprising a negatively-charged tag segment heterologous to the rest of the polypeptide cargo and at the N-terminus or the C-terminus of the polypeptide cargo, wherein the tag segment binds to the binding segment of the carrier polypeptide through an electrostatic interaction. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBK10. In some embodiments, the cell-targeting segment is Internalin B or a variant thereof. In some embodiments, the carrier polypeptide is InlBPBK10. In some embodiments, the cell-targeting segment is hepatocyte growth factor (HGF) or a variant thereof. In some embodiments, the carrier polypeptide is HGFPBK10. In some embodiments, the tag segment is about 4 amino acids to about 20 amino acids in length. In some embodiments, the polypeptide cargo comprises a therapeutic polypeptide, such as a cytotoxic polypeptide. In some embodiments, the cytotoxic polypeptide comprises a protein-synthesis inhibitor, such as gelonin or a variant thereof. In some embodiments, the polypeptide cargo is about 5 kDa to about 50 kDa. In some embodiments, the polypeptide cargo is less than about 5 kDa. In some embodiments, the molar ratio of the carrier polypeptide to the polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, a composition comprises nanoparticles comprising a carrier polypeptide comprising a penton base segment and a negatively-charged binding segment; and a polypeptide cargo comprising a positively-charged tag segment heterologous to the rest of the polypeptide cargo and at the N-terminus or the C-terminus of the polypeptide cargo, wherein the tag segment binds to the binding segment of the carrier polypeptide through an electrostatic interaction. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBD10. In some embodiments, the cell-targeting segment is Internalin B or a variant thereof. In some embodiments, the carrier polypeptide is InlBPBD10. In some embodiments, the cell-targeting segment is hepatocyte growth factor (HGF) or a variant thereof. In some embodiments, the carrier polypeptide is HGFPBD10. In some embodiments, the tag segment is about 4 amino acids to about 20 amino acids in length. In some embodiments, the polypeptide cargo comprises a therapeutic polypeptide, such as a cytotoxic polypeptide. In some embodiments, the cytotoxic polypeptide comprises a protein-synthesis inhibitor, such as gelonin or a variant thereof. In some embodiments, the polypeptide cargo is about 5 kDa to about 50 kDa. In some embodiments, the polypeptide cargo is less than about 5 kDa. In some embodiments, the molar ratio of the carrier polypeptide to the polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, a composition comprises nanoparticles comprising a carrier polypeptide comprising a penton base segment and a positively-charged binding segment comprising poly-lysine (such as deca-lysine) or poly-arginine; and a polypeptide cargo comprising a negatively-charged tag segment heterologous to the rest of the polypeptide cargo and at the N-terminus or the C-terminus of the polypeptide cargo, where the tag segment comprises poly-aspatic acid (such as deca-aspartic acid) or poly-glutamic acid, and wherein the tag segment binds to the binding segment of the carrier polypeptide through an electrostatic interaction. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBK10. In some embodiments, the cell-targeting segment is Internalin B or a variant thereof. In some embodiments, the carrier polypeptide is InlBPBK10. In some embodiments, the cell-targeting segment is hepatocyte growth factor (HGF) or a variant thereof. In some embodiments, the carrier polypeptide is HGFPBK10. In some embodiments, the tag segment is about 4 amino acids to about 20 amino acids in length. In some embodiments, the polypeptide cargo comprises a therapeutic polypeptide, such as a cytotoxic polypeptide. In some embodiments, the cytotoxic polypeptide comprises a protein-synthesis inhibitor, such as gelonin or a variant thereof. In some embodiments, the polypeptide cargo is about 5 kDa to about 50 kDa. In some embodiments, the polypeptide cargo is less than about 5 kDa. In some embodiments, the molar ratio of the carrier polypeptide to the polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, a composition comprises nanoparticles comprising a carrier polypeptide comprising a penton base segment and a negatively-charged binding segment comprising poly-aspartic acid (such as deca-aspartic acid) or poly-glutamic acid; and a polypeptide cargo comprising a positively-charged tag segment heterologous to the rest of the polypeptide cargo and at the N-terminus or the C-terminus of the polypeptide cargo, where the tag segment comprises poly-lysine (such as deca-lysine) or poly-arginine, and wherein the tag segment binds to the binding segment of the carrier polypeptide through an electrostatic interaction. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBD10. In some embodiments, the cell-targeting segment is Internalin B or a variant thereof. In some embodiments, the carrier polypeptide is InlBPBD10. In some embodiments, the cell-targeting segment is hepatocyte growth factor (HGF) or a variant thereof. In some embodiments, the carrier polypeptide is HGFPBD10. In some embodiments, the tag segment is about 4 amino acids to about 20 amino acids in length. In some embodiments, the polypeptide cargo comprises a therapeutic polypeptide, such as a cytotoxic polypeptide. In some embodiments, the cytotoxic polypeptide comprises a protein-synthesis inhibitor, such as gelonin or a variant thereof. In some embodiments, the polypeptide cargo is about 5 kDa to about 50 kDa. In some embodiments, the polypeptide cargo is less than about 5 kDa. In some embodiments, the molar ratio of the carrier polypeptide to the polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, a composition comprises nanoparticles comprising a carrier polypeptide comprising a penton base segment and a negatively-charged binding segment that binds to a positively-charged cargo through an electrostatic interaction. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBD10. In some embodiments, the cell-targeting segment is Internalin B or a variant thereof. In some embodiments, the carrier poly peptide is InlBPBD10. In some embodiments, the cell-targeting segment is hepatocyte growth factor (HGF) or a variant thereof. In some embodiments, the carrier polypeptide is HGFPBD10. In some embodiments, the molar ratio of the carrier polypeptide to the cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, a composition comprises nanoparticles comprising a carrier polypeptide comprising a penton base segment and a negatively-charged binding segment that binds to a positively-charged polypeptide cargo through an electrostatic interaction. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBD10. In some embodiments, the cell-targeting segment is Internalin B or a variant thereof. In some embodiments, the carrier polypeptide is InlBPBD10. In some embodiments, the cell-targeting segment is hepatocyte growth factor (HGF) or a variant thereof. In some embodiments, the carrier polypeptide is HGFPBD10. In some embodiments, the polypeptide cargo comprises a therapeutic polypeptide, such as a cytotoxic polypeptide. In some embodiments, the cytotoxic polypeptide comprises a protein-synthesis inhibitor, such as gelonin or a variant thereof. In some embodiments, the polypeptide cargo is about 5 kDa to about 50 kDa. In some embodiments, the polypeptide cargo is less than about 5 kDa. In some embodiments, the molar ratio of the carrier polypeptide to the polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, a composition comprises nanoparticles comprising a carrier polypeptide comprising a penton base segment and a negatively-charged binding segment comprising poly-aspartic acid (such as deca-aspartic acid) or poly-glutamic acid that binds to a positively-charged polypeptide cargo through an electrostatic interaction. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBD10. In some embodiments, the cell-targeting segment is Internalin B or a variant thereof. In some embodiments, the carrier polypeptide is InlBPBD10. In some embodiments, the cell-targeting segment is hepatocyte growth factor (HGF) or a variant thereof. In some embodiments, the carrier polypeptide is HGFPBD10. In some embodiments, the polypeptide cargo comprises a therapeutic polypeptide, such as a cytotoxic polypeptide. In some embodiments, the cytotoxic polypeptide comprises a protein-synthesis inhibitor, such as gelonin or a variant thereof. In some embodiments, the polypeptide cargo is about 5 kDa to about 50 kDa. In some embodiments, the polypeptide cargo is less than about 5 kDa. In some embodiments, the molar ratio of the carrier polypeptide to the polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, a method of making a nanoparticle composition comprises combining a carrier polypeptide with a polypeptide cargo, the carrier polypeptide comprising a penton base segment and a binding segment, and the polypeptide cargo comprising a tag segment that binds to the binding segment of the carrier polypeptide through an electrostatic interaction. In some embodiments, the method further comprises sterile filtering the composition. In some embodiments, the method further comprises dispensing the composition in a vial. In some embodiments, the method further comprises lyophilizing the composition. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBK10. In some embodiments, the cell-targeting segment is Internalin B or a variant thereof. In some embodiments, the carrier polypeptide is InlBPBK10. In some embodiments, the cell-targeting segment is hepatocyte growth factor (HGF) or a variant thereof. In some embodiments, the carrier polypeptide is HGFPBK10. In some embodiments, the tag segment is about 4 amino acids to about 20 amino acids in length. In some embodiments, the polypeptide cargo comprises a therapeutic polypeptide, such as a cytotoxic polypeptide. In some embodiments, the cytotoxic polypeptide comprises a protein-synthesis inhibitor, such as gelonin or a variant thereof. In some embodiments, the polypeptide cargo is about 5 kDa to about 50 kDa. In some embodiments, the polypeptide cargo is less than about 5 kDa. In some embodiments, the molar ratio of the carrier polypeptide to the polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, a method of making a nanoparticle composition comprises combining a carrier polypeptide with a polypeptide cargo, the carrier polypeptide comprising a penton base segment and a binding segment, and the polypeptide cargo comprising a tag segment heterologous to the rest of the polypeptide cargo that binds to the binding segment of the carrier polypeptide through an electrostatic interaction. In some embodiments, the method further comprises sterile filtering the composition. In some embodiments, the method further comprises dispensing the composition in a vial. In some embodiments, the method further comprises lyophilizing the composition. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBK10. In some embodiments, the cell-targeting segment is Internalin B or a variant thereof. In some embodiments, the carrier polypeptide is InlBPBK10. In some embodiments, the cell-targeting segment is hepatocyte growth factor (HGF) or a variant thereof. In some embodiments, the carrier polypeptide is HGFPBK10. In some embodiments, the tag segment is about 4 amino acids to about 20 amino acids in length. In some embodiments, the polypeptide cargo comprises a therapeutic polypeptide, such as a cytotoxic polypeptide. In some embodiments, the cytotoxic polypeptide comprises a protein-synthesis inhibitor, such as gelonin or a variant thereof. In some embodiments, the polypeptide cargo is about 5 kDa to about 50 kDa. In some embodiments, the polypeptide cargo is less than about 5 kDa. In some embodiments, the molar ratio of the carrier polypeptide to the polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, a method of making a nanoparticle composition comprises combining a carrier polypeptide with a polypeptide cargo, the carrier polypeptide comprising a penton base segment and a binding segment, and the polypeptide cargo comprising a tag segment heterologous to the rest of the polypeptide cargo and at the N-terminus or the C-terminus of the polypeptide cargo, wherein the tag segment of the polypeptide cargo binds to the binding segment of the carrier polypeptide through an electrostatic interaction. In some embodiments, the method further comprises sterile filtering the composition. In some embodiments, the method further comprises dispensing the composition in a vial. In some embodiments, the method further comprises lyophilizing the composition. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBK10. In some embodiments, the cell-targeting segment is Internalin B or a variant thereof. In some embodiments, the carrier polypeptide is InlBPBK10. In some embodiments, the cell-targeting segment is hepatocyte growth factor (HGF) or a variant thereof. In some embodiments, the carrier polypeptide is HGFPBK10. In some embodiments, the tag segment is about 4 amino acids to about 20 amino acids in length. In some embodiments, the polypeptide cargo comprises a therapeutic polypeptide, such as a cytotoxic polypeptide. In some embodiments, the cytotoxic polypeptide comprises a protein-synthesis inhibitor, such as gelonin or a variant thereof. In some embodiments, the polypeptide cargo is about 5 kDa to about 50 kDa. In some embodiments, the polypeptide cargo is less than about 5 kDa. In some embodiments, the molar ratio of the carrier polypeptide to the polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, a method of making a nanoparticle composition comprises combining a carrier polypeptide with a polypeptide cargo, the carrier polypeptide comprising a penton base segment and a positively-charged binding segment, and the polypeptide cargo comprising a negatively-charged tag segment heterologous to the rest of the polypeptide cargo and at the N-terminus or the C-terminus of the polypeptide cargo, wherein the tag segment of the polypeptide cargo binds to the binding segment of the carrier polypeptide through an electrostatic interaction. In some embodiments, the method further comprises sterile filtering the composition. In some embodiments, the method further comprises dispensing the composition in a vial. In some embodiments, the method further comprises lyophilizing the composition. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBK10. In some embodiments, the cell-targeting segment is Internalin B or a variant thereof. In some embodiments, the carrier polypeptide is InlBPBK10. In some embodiments, the cell-targeting segment is hepatocyte growth factor (HGF) or a variant thereof. In some embodiments, the carrier polypeptide is HGFPBK10. In some embodiments, the tag segment is about 4 amino acids to about 20 amino acids in length. In some embodiments, the polypeptide cargo comprises a therapeutic polypeptide, such as a cytotoxic polypeptide. In some embodiments, the cytotoxic polypeptide comprises a protein-synthesis inhibitor, such as gelonin or a variant thereof. In some embodiments, the polypeptide cargo is about 5 kDa to about 50 kDa. In some embodiments, the polypeptide cargo is less than about 5 kDa. In some embodiments, the molar ratio of the carrier polypeptide to the polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, a method of making a nanoparticle composition comprises combining a carrier polypeptide with a polypeptide cargo, the carrier polypeptide comprising a penton base segment and a negatively-charged binding segment, and the polypeptide cargo comprising a positively-charged tag segment heterologous to the rest of the polypeptide cargo and at the N-terminus or the C-terminus of the polypeptide cargo, wherein the tag segment of the polypeptide cargo binds to the binding segment of the carrier polypeptide through an electrostatic interaction. In some embodiments, the method further comprises sterile filtering the composition. In some embodiments, the method further comprises sterile filtering the composition. In some embodiments, the method further comprises dispensing the composition in a vial. In some embodiments, the method further comprises lyophilizing the composition. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBD10. In some embodiments, the cell-targeting segment is Internalin B or a variant thereof. In some embodiments, the carrier polypeptide is InlBPBD10. In some embodiments, the cell-targeting segment is hepatocyte growth factor (HGF) or a variant thereof. In some embodiments, the carrier polypeptide is HGFPBD10. In some embodiments, the tag segment is about 4 amino acids to about 20 amino acids in length. In some embodiments, the polypeptide cargo comprises a therapeutic polypeptide, such as a cytotoxic polypeptide. In some embodiments, the cytotoxic polypeptide comprises a protein-synthesis inhibitor, such as gelonin or a variant thereof. In some embodiments, the polypeptide cargo is about 5 kDa to about 50 kDa. In some embodiments, the polypeptide cargo is less than about 5 kDa. In some embodiments, the molar ratio of the carrier polypeptide to the polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, a method of making a nanoparticle composition comprises combining a carrier polypeptide with a polypeptide cargo, the carrier polypeptide comprising a penton base segment and a positively-charged binding segment comprising poly-lysine (such as deca-lysine) or poly-arginine, and the polypeptide cargo comprising a negatively-charged tag segment heterologous to the rest of the polypeptide cargo and at the N-terminus or the C-terminus of the polypeptide cargo, wherein the tag segment comprises poly-aspartic acid (such as deca-aspartic acid) or poly-glutamic acid, and wherein the tag segment of the polypeptide cargo binds to the binding segment of the carrier polypeptide through an electrostatic interaction. In some embodiments, the method further comprises sterile filtering the composition. In some embodiments, the method further comprises dispensing the composition in a vial. In some embodiments, the method further comprises lyophilizing the composition. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBK10. In some embodiments, the cell-targeting segment is Internalin B or a variant thereof. In some embodiments, the carrier polypeptide is InlBPBK10. In some embodiments, the cell-targeting segment is hepatocyte growth factor (HGF) or a variant thereof. In some embodiments, the carrier polypeptide is HGFPBK10. In some embodiments, the tag segment is about 4 amino acids to about 20 amino acids in length. In some embodiments, the polypeptide cargo comprises a therapeutic polypeptide, such as a cytotoxic polypeptide. In some embodiments, the cytotoxic polypeptide comprises a protein-synthesis inhibitor, such as gelonin or a variant thereof. In some embodiments, the polypeptide cargo is about 5 kDa to about 50 kDa. In some embodiments, the polypeptide cargo is less than about 5 kDa. In some embodiments, the molar ratio of the carrier polypeptide to the polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, a method of making a nanoparticle composition comprises combining a carrier polypeptide with a polypeptide cargo, the carrier polypeptide comprising a penton base segment and a positively-charged binding segment comprising poly-aspartic acid (such as deca-aspartic acid) or poly-glutamic acid, and the polypeptide cargo comprising a negatively-charged tag segment heterologous to the rest of the polypeptide cargo and at the N-terminus or the C-terminus of the polypeptide cargo, wherein the tag segment comprises poly-lysine (such as deca-lysine) or poly-arginine, and wherein the tag segment of the polypeptide cargo binds to the binding segment of the carrier polypeptide through an electrostatic interaction. In some embodiments, the method further comprises sterile filtering the composition. In some embodiments, the method further comprises dispensing the composition in a vial. In some embodiments, the method further comprises lyophilizing the composition. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBD10. In some embodiments, the cell-targeting segment is Internalin B or a variant thereof. In some embodiments, the carrier polypeptide is InlBPBD10. In some embodiments, the cell-targeting segment is hepatocyte growth factor (HGF) or a variant thereof. In some embodiments, the carrier polypeptide is HGFPBD10. In some embodiments, the tag segment is about 4 amino acids to about 20 amino acids in length. In some embodiments, the polypeptide cargo comprises a therapeutic polypeptide, such as a cytotoxic polypeptide. In some embodiments, the cytotoxic polypeptide comprises a protein-synthesis inhibitor, such as gelonin or a variant thereof. In some embodiments, the polypeptide cargo is about 5 kDa to about 50 kDa. In some embodiments, the polypeptide cargo is less than about 5 kDa. In some embodiments, the molar ratio of the carrier polypeptide to the polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, a method of making a nanoparticle composition comprises combining a carrier polypeptide comprising a penton base segment and a negatively-charged binding segment with a positively-charged cargo, wherein the binding segment of the carrier polypeptide binds to the positively-charged cargo through an electrostatic interaction. In some embodiments, the method further comprises sterile filtering the composition. In some embodiments, the method further comprises dispensing the composition in a vial. In some embodiments, the method further comprises lyophilizing the composition. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBD10. In some embodiments, the cell-targeting segment is Internalin B or a variant thereof. In some embodiments, the carrier polypeptide is InlBPBD10. In some embodiments, the cell-targeting segment is hepatocyte growth factor (HGF) or a variant thereof. In some embodiments, the carrier polypeptide is HGFPBD10. In some embodiments, the molar ratio of the carrier polypeptide to the polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, a method of making a nanoparticle composition comprises combining a carrier polypeptide comprising a penton base segment and a negatively-charged binding segment with a positively-charged polypeptide cargo, wherein the binding segment of the carrier polypeptide binds to the positively-charged polypeptide cargo through an electrostatic interaction. In some embodiments, the method further comprises sterile filtering the composition. In some embodiments, the method further comprises dispensing the composition in a vial. In some embodiments, the method further comprises lyophilizing the composition. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBD10. In some embodiments, the cell-targeting segment is Internalin B or a variant thereof. In some embodiments, the carrier polypeptide is InlBPBD10. In some embodiments, the cell-targeting segment is hepatocyte growth factor (HGF) or a variant thereof. In some embodiments, the carrier polypeptide is HGFPBD10. In some embodiments, the polypeptide cargo comprises a therapeutic polypeptide, such as a cytotoxic polypeptide. In some embodiments, the cytotoxic polypeptide comprises a protein-synthesis inhibitor, such as gelonin or a variant thereof. In some embodiments, the polypeptide cargo is about 5 kDa to about 50 kDa. In some embodiments, the polypeptide cargo is less than about 5 kDa. In some embodiments, the molar ratio of the carrier polypeptide to the polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, a method of making a nanoparticle composition comprises combining a carrier polypeptide comprising a penton base segment and a poly-aspartic acid (such as deca-aspartic acid) or poly-glutamic acid binding segment with a positively-charged polypeptide cargo, wherein the binding segment of the carrier polypeptide binds to the positively-charged polypeptide cargo through an electrostatic interaction. In some embodiments, the method further comprises sterile filtering the composition. In some embodiments, the method further comprises dispensing the composition in a vial. In some embodiments, the method further comprises lyophilizing the composition. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBD10. In some embodiments, the cell-targeting segment is Internalin B or a variant thereof. In some embodiments, the carrier polypeptide is InlBPBD10. In some embodiments, the cell-targeting segment is hepatocyte growth factor (HGF) or a variant thereof. In some embodiments, the carrier polypeptide is HGFPBD10. In some embodiments, the polypeptide cargo comprises a therapeutic polypeptide, such as a cytotoxic polypeptide. In some embodiments, the cytotoxic polypeptide comprises a protein-synthesis inhibitor, such as gelonin or a variant thereof. In some embodiments, the polypeptide cargo is about 5 kDa to about 50 kDa. In some embodiments, the polypeptide cargo is less than about 5 kDa. In some embodiments, the molar ratio of the carrier polypeptide to the polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

The nanoparticles in the compositions described herein can be useful for delivery a cargo to a cell. In some embodiments, a method of delivering a polypeptide cargo to a cell comprises contacting the cell with the composition comprising nanoparticles comprising a carrier polypeptide and a polypeptide cargo. In some embodiments, a method of delivery a polypeptide cargo to a cell comprises contacting the cell with a nanoparticle comprising a carrier polypeptide and a polypeptide cargo.

In some embodiments, a method of delivering a polypeptide cargo to a cell comprises contact the cell with a nanoparticle comprising a carrier polypeptide comprising a penton base segment and a binding segment; and the polypeptide cargo, the polypeptide cargo comprising a tag segment that binds to the binding segment of the carrier polypeptide through an electrostatic interaction. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBK10. In some embodiments, the cell-targeting segment is Internalin B or a variant thereof. In some embodiments, the carrier polypeptide is InlBPBK10. In some embodiments, the cell-targeting segment is hepatocyte growth factor (HGF) or a variant thereof. In some embodiments, the carrier polypeptide is HGFPBK10. In some embodiments, the tag segment is about 4 amino acids to about 20 amino acids in length. In some embodiments, the polypeptide cargo comprises a therapeutic polypeptide, such as a cytotoxic polypeptide. In some embodiments, the cytotoxic polypeptide comprises a protein-synthesis inhibitor, such as gelonin or a variant thereof. In some embodiments, the polypeptide cargo is about 5 kDa to about 50 kDa. In some embodiments, the polypeptide cargo is less than about 5 kDa. In some embodiments, the molar ratio of the carrier polypeptide to the polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

### Pharmaceutical Compositions

In some embodiments, the nanoparticle compositions described herein are formulated as pharmaceutical compositions comprising a plurality of nanoparticles described herein and a pharmaceutically acceptable excipient.

In some embodiments, the pharmaceutical composition is a solid, such as a powder. The powder can be formed, for example, by lyophilizing the nanoparticles in solution. The powder can be reconstituted, for example by mixing the powder with an aqueous liquid (e.g., water or a buffer). In some embodiments, the pharmaceutical composition is a liquid, for example nanoparticles suspended in an aqueous solution (such as physiological saline or Ringer's solution). In some embodiments, the pharmaceutical composition comprises a pharmaceutically-acceptable excipient, for example a filler, binder, coating, preservative, lubricant, flavoring agent, sweetening agent, coloring agent, a solvent, a buffering agent, a chelating agent, or stabilizer.

Examples of pharmaceutically-acceptable fillers include cellulose, dibasic calcium phosphate, calcium carbonate, microcrystalline cellulose, sucrose, lactose, glucose, mannitol, sorbitol, maltol, pregelatinized starch, corn starch, or potato starch. Examples of pharmaceutically-acceptable binders include polyvinylpyrrolidone, starch, lactose, xylitol, sorbitol, maltitol, gelatin, sucrose, polyethylene glycol, methyl cellulose, or cellulose. Examples of pharmaceutically-acceptable coatings include hydroxypropyl methylcellulose (HPMC), shellac, corn protein zein, or gelatin. Examples of pharmaceutically-acceptable disintegrants include polyvinylpyrrolidone, carboxymethyl cellulose, or sodium starch glycolate. Examples of pharmaceutically-acceptable lubricants include polyethylene glycol, magnesium stearate, or stearic acid. Examples of pharmaceutically-acceptable preservatives include methyl parabens, ethyl parabens, propyl paraben, benzoic acid, or sorbic acid. Examples of pharmaceutically-acceptable sweetening agents include sucrose, saccharine, aspartame, or sorbitol. Examples of pharmaceutically-acceptable buffering agents include carbonates, citrates, gluconates, acetates, phosphates, or tartrates.

### Treatments of Disease

Nanoparticle compositions can be useful for the treatment of disease (such as cancer) in a subject by administering an effective amount of a composition comprising the nanoparticles to the subject. In some embodiments, the method kills diseased cells (e.g., cancer cells). In some embodiments, the cell-targeting segment of the carrier polypeptide binds to a molecule on the surface of a diseased cell, thereby delivering the cargo (e.g., a cargo polypeptide or positively charged cargo) to the diseased cells. The carrier polypeptide and the cargo can then enter the cell via endosomes. The penton base segment allows for endosomal escape of the carrier polypeptide and cargo, wherein the carrier polypeptide and cargo enter the cell. In some embodiments, the carrier polypeptide and cargo is trafficked to the nucleus of the cell, for example if the penton base comprises one or more variants than enhance localization of the carrier polypeptide to the nucleus of the cell. Once in the cell, the carrier polypeptide and the cargo can separate, and the cargo can perform an intended function (such as kill the cell).

In some embodiments, a method of treating a disease in a subject comprises administering to the subject with the disease an effective amount of a composition comprising nanoparticles to the subject, the nanoparticles comprising a carrier polypeptide comprising a penton base segment and a binding segment, and a polypeptide cargo comprising a tag segment that binds to the binding segment of the carrier polypeptide through an electrostatic interaction.

In some embodiments, a method of treating a disease in a subject comprises administering to the subject with the disease an effective amount of a composition comprising nanoparticles to the subject, the nanoparticles comprising a carrier polypeptide comprising a penton base segment and a negatively-charged binding segment bound to a positively-charged cargo through an electrostatic interaction.

In some embodiments, a method of treating a cancer in a subject comprises administering to the subject with the cancer an effective amount of a composition comprising nanoparticles to the subject, the nanoparticles comprising a carrier polypeptide comprising a penton base segment and a binding segment, and a cytotoxic polypeptide cargo comprising a tag segment that binds to the binding segment of the carrier polypeptide through an electrostatic interaction. In some embodiments, the carrier polypeptide targets a cancer cell (for example, by further comprising a cell-targeting segment that targets a cancer cell, such as a HER3+ cancer cell or a c-MET+ cancer cell). In some embodiments, the cancer is metastatic.

In some embodiments, a method of treating a cancer in a subject comprises administering to the subject with the cancer an effective amount of a composition comprising nanoparticles to the subject, the nanoparticles comprising a carrier polypeptide comprising a penton base segment and a negatively-charged binding segment bound to a positively-charged cytotoxic cargo through an electrostatic interaction. In some embodiments, the cytotoxic cargo comprises a cytotoxic polypeptide cargo. In some embodiments, the carrier polypeptide targets a cancer cell (for example, by further comprising a cell-targeting segment that targets a cancer cell, such as a HER3+ cancer cell or a c-MET+ cancer cell). In some embodiments, the cancer is metastatic.

In some embodiments, the cancer is a HER3+ cancer. A Her cell-targeting segment, for example, can bind HER3 present on the surface of the HER3+ cancer cells to target the nanoparticles to the cancer cells. In some embodiments, the cancer is a c-MET+ cancer. An InlB cell-targeting segment, for example, can bind c-MET present on the surface of the c-MET+ cancer cell to target the nanoparticles to the cancer cells.

In some embodiments, an effective amount of a composition comprising the nanoparticles is administered to subject to treat a head and neck cancer, a pancreatic cancer, a breast cancer, an ovarian cancer, a glial cancer, a cervical cancer, a gastric cancer, a skin cancer, a colon cancer, a rectal cancer, a lung cancer, a kidney cancer, or a thyroid cancer. In some embodiments, the cancer is a triple negative breast cancer. Many cancers exhibit upregulated expression for a particular cell surface molecule. One or more of such upregulated molecules are preferred targets for the cell-targeting segment of the carrier protein.

In some embodiments, the method of treating a subject with cancer further comprises a secondary therapy, such as radiation therapy or surgery. Thus, in some embodiments, the composition comprising the nanoparticles described herein is administered to a subject with cancer as a neoadjuvant therapy.

In some embodiments, the subject has not undergone chemotherapy or radiation therapy prior to administration of the nanoparticles described herein. In some embodiments, the subject has undergone chemotherapy or radiation therapy.

In some embodiments, the nanoparticle composition described herein is administered to a subject. In some embodiments, the nanoparticle composition is administered to a subject for in vivo delivery to targeted cells. Generally, dosages and routes of administration of the nanoparticle composition are determined according to the size and condition of the subject, according to standard pharmaceutical practice. In some embodiments, the nanoparticle composition is administered to a subject through any route, including orally, transdermally, by inhalation, intravenously, intra-arterially, intramuscularly, direct application to a wound site, application to a surgical site, intraperitoneally, by suppository, subcutaneously, intradermally, transcutaneously, by nebulization, intrapleurally, intraventricularly, intra-articularly, intraocularly, or intraspinally. In some embodiments, the composition is administered to a subject intravenously.

In some embodiments, the method further comprises administering an additional therapy, such as an anticancer therapy, in combination with administering the nanoparticle composition to the subject. The additional therapy can be an adjuvant or a neoadjuvant to the administered nanoparticles. See, for example U.S. Published Patent Application US 20160/060316. In some embodiments, the additional therapy is administered prior to administering the nanoparticle composition. In some embodiments, the additional therapy is administered after administering the nanoparticle composition. In some embodiments, the additional therapy is administered contemporaneous (i.e., simultaneously or approximately simultaneously) to administering the nanoparticle composition. In some embodiments, the additional therapy comprises administering a HER2 antibody to the subject. In some embodiments, the HER2 antibody is trastuzumab, pertuzumab, or a combination thereof. In some embodiments, the additional therapy comprises administering a HER2 inhibitor. In some embodiments, the HER2 inhibitor is lapatinib.

In some embodiments, the method of treating a cancer in a subject comprises administering an effective amount of a composition comprising nanoparticles comprising a carrier polypeptide comprising a penton base segment and a binding segment; and a cytotoxic polypeptide cargo comprising a tag segment bound to the binding segment of the carrier polypeptide through an electrostatic interaction. In some embodiments, the carrier polypeptide targets a cancer cell that overexpresses integrin. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBK10. In some embodiments, the cell-targeting segment is Internalin B or a variant thereof. In some embodiments, the carrier polypeptide is InlBPBK10. In some embodiments, the cell-targeting segment is hepatocyte growth factor (HGF) or a variant thereof. In some embodiments, the carrier polypeptide is HGFPBK10. In some embodiments, the tag segment is about 4 amino acids to about 20 amino acids in length. In some embodiments, the cytotoxic polypeptide cargo comprises a protein-synthesis inhibitor, such as gelonin or a variant thereof. In some embodiments, the cytotoxic polypeptide cargo is about 5 kDa to about 50 kDa. In some embodiments, the cytotoxic polypeptide cargo is less than about 5 kDa. In some embodiments, the molar ratio of the carrier polypeptide to the cytotoxic polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, the method of treating a cancer in a subject comprises administering an effective amount of a composition comprising nanoparticles comprising a carrier polypeptide comprising a penton base segment and a binding segment; and a cytotoxic polypeptide cargo comprising a tag segment heterologous to the rest of the polypeptide cargo that binds to the binding segment of the carrier polypeptide through an electrostatic interaction. In some embodiments, the carrier polypeptide targets a cancer cell that overexpresses integrin. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBK10. In some embodiments, the cell-targeting segment is Internalin B or a variant thereof. In some embodiments, the carrier polypeptide is InlBPBK10. In some embodiments, the cell-targeting segment is hepatocyte growth factor (HGF) or a variant thereof. In some embodiments, the carrier polypeptide is HGFPBK10. In some embodiments, the tag segment is about 4 amino acids to about 20 amino acids in length. In some embodiments, the cytotoxic polypeptide cargo comprises a protein-synthesis inhibitor, such as gelonin or a variant thereof. In some embodiments, the cytotoxic polypeptide cargo is about 5 kDa to about 50 kDa. In some embodiments, the cytotoxic polypeptide cargo is less than about 5 kDa. In some embodiments, the molar ratio of the carrier polypeptide to the cytotoxic polypeptide cargo is about 3:1 to about 8:1. In some embodiments, the average size of the nanoparticles in the composition is about 100 nm or less.

In some embodiments, the method of treating a cancer in a subject comprises administering an effective amount of a composition comprising nanoparticles comprising a carrier polypeptide comprising a penton base segment and a binding segment; and a cytotoxic polypeptide cargo comprising a tag segment heterologous to the rest of the polypeptide cargo and at the N-terminus or the C-terminus of the polypeptide polypeptide cargo, wherein the tag segment binds to the binding segment of the carrier polypeptide through an electrostatic interaction. In some embodiments, the carrier polypeptide targets a cancer cell that overexpresses integrin. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBK10. In some embodiments, the cell-targeting segment is Internalin B or a variant thereof. In some embodiments, the carrier polypeptide is InlBPBK10. In some embodiments, the cell-targeting segment is hepatocyte growth factor (HGF) or a variant thereof. In some embodiments, the carrier polypeptide is HGFPBK10. In some embodiments, the tag segment is about 4 amino acids to about 20 amino acids in length. In some embodiments, the cytotoxic polypeptide cargo comprises a protein-synthesis inhibitor, such as gelonin or a variant thereof. In some embodiments, the cytotoxic polypeptide cargo is about 5 kDa to about 50 kDa. In some embodiments, the cytotoxic polypeptide cargo is less than about 5 kDa. In some embodiments, the molar ratio of the carrier polypeptide to the cytotoxic polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, the method of treating a cancer in a subject comprises administering an effective amount of a composition comprising nanoparticles comprising a carrier polypeptide comprising a penton base segment and a positively-charged binding segment; and a cytotoxic polypeptide cargo comprising a negatively-charged tag segment heterologous to the rest of the cytotoxic polypeptide cargo and at the N-terminus or the C-terminus of the cytotoxic polypeptide cargo, wherein the tag segment binds to the binding segment of the carrier polypeptide through an electrostatic interaction. In some embodiments, the carrier polypeptide targets a cancer cell that overexpresses integrin. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBK10. In some embodiments, the cell-targeting segment is Internalin B or a variant thereof. In some embodiments, the carrier polypeptide is InlBPBK10. In some embodiments, the cell-targeting segment is hepatocyte growth factor (HGF) or a variant thereof. In some embodiments, the carrier polypeptide is HGFPBK10. In some embodiments, the tag segment is about 4 amino acids to about 20 amino acids in length. In some embodiments, the cytotoxic polypeptide cargo comprises a protein-synthesis inhibitor, such as gelonin or a variant thereof. In some embodiments, the cytotoxic polypeptide cargo is about 5 kDa to about 50 kDa. In some embodiments, the cytotoxic polypeptide cargo is less than about 5 kDa. In some embodiments, the molar ratio of the carrier polypeptide to the cytotoxic polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, the method of treating a cancer in a subject comprises administering an effective amount of a composition comprising nanoparticles comprising a carrier polypeptide comprising a penton base segment and a negatively-charged binding segment; and a cytotoxic polypeptide cargo comprising a positively-charged tag segment heterologous to the rest of the cytotoxic polypeptide cargo and at the N-terminus or the C-terminus of the cytotoxic polypeptide cargo, wherein the tag segment binds to the binding segment of the carrier polypeptide through an electrostatic interaction. In some embodiments, the carrier polypeptide targets a cancer cell that overexpresses integrin. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBD10. In some embodiments, the cell-targeting segment is Internalin B or a variant thereof. In some embodiments, the carrier polypeptide is InlBPBD10. In some embodiments, the cell-targeting segment is hepatocyte growth factor (HGF) or a variant thereof. In some embodiments, the carrier polypeptide is HGFPBD10. In some embodiments, the tag segment is about 4 amino acids to about 20 amino acids in length. In some embodiments, the cytotoxic polypeptide cargo comprises a protein-synthesis inhibitor, such as gelonin or a variant thereof. In some embodiments, the cytotoxic polypeptide cargo is about 5 kDa to about 50 kDa. In some embodiments, the cytotoxic polypeptide cargo is less than about 5 kDa. In some embodiments, the molar ratio of the carrier polypeptide to the cytotoxic polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, the method of treating a cancer in a subject comprises administering an effective amount of a composition comprising nanoparticles comprising a carrier polypeptide comprising a penton base segment and a positively-charged binding segment comprising poly-lysine (such as deca-lysine) or poly-arginine; and a cytotoxic polypeptide cargo comprising a negatively-charged tag segment heterologous to the rest of the cytotoxic polypeptide cargo and at the N-terminus or the C-terminus of the polypeptide cargo, where the tag segment comprises poly-aspatic acid (such as deca-aspartic acid) or poly-glutamic acid, and wherein the tag segment binds to the binding segment of the carrier polypeptide through an electrostatic interaction. In some embodiments, the carrier polypeptide targets a cancer cell that overexpresses integrin. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBK10. In some embodiments, the cell-targeting segment is Internalin B or a variant thereof. In some embodiments, the carrier polypeptide is InlBPBK10. In some embodiments, the cell-targeting segment is hepatocyte growth factor (HGF) or a variant thereof. In some embodiments, the carrier polypeptide is HGFPBK10. In some embodiments, the tag segment is about 4 amino acids to about 20 amino acids in length. In some embodiments, the cytotoxic polypeptide cargo comprises a protein-synthesis inhibitor, such as gelonin or a variant thereof. In some embodiments, the cytotoxic polypeptide cargo is about 5 kDa to about 50 kDa. In some embodiments, the cytotoxic polypeptide cargo is less than about 5 kDa. In some embodiments, the molar ratio of the carrier polypeptide to the cytotoxic polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, the method of treating a cancer in a subject comprises administering an effective amount of a composition comprising nanoparticles comprising a carrier polypeptide comprising a penton base segment and a negatively-charged binding segment comprising poly-aspartic acid (such as deca-aspartic acid) or poly-glutamic acid; and a cytotoxic polypeptide cargo comprising a positively-charged tag segment heterologous to the rest of the cytotoxic polypeptide cargo and at the N-terminus or the C-terminus of the cytotoxic polypeptide cargo, where the tag segment comprises poly-lysine (such as deca-lysine) or poly-arginine, and wherein the tag segment binds to the binding segment of the carrier polypeptide through an electrostatic interaction. In some embodiments, the carrier polypeptide targets a cancer cell that overexpresses integrin. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBD10. In some embodiments, the cell-targeting segment is Internalin B or a variant thereof. In some embodiments, the carrier polypeptide is InlBPBD10. In some embodiments, the cell-targeting segment is hepatocyte growth factor (HGF) or a variant thereof. In some embodiments, the carrier polypeptide is HGFPBD10. In some embodiments, the tag segment is about 4 amino acids to about 20 amino acids in length. In some embodiments, the cytotoxic polypeptide cargo comprises a protein-synthesis inhibitor, such as gelonin or a variant thereof. In some embodiments, the cytotoxic polypeptide cargo is about 5 kDa to about 50 kDa. In some embodiments, the cytotoxic polypeptide cargo is less than about 5 kDa. In some embodiments, the molar ratio of the carrier polypeptide to the cytotoxic polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, the method of treating a cancer in a subject comprises administering an effective amount of a composition comprising nanoparticles comprising a carrier polypeptide comprising a penton base segment and a negatively-charged binding segment bound to a positively-charged cytotoxic cargo through an electrostatic interaction. In some embodiments, the carrier polypeptide targets a cancer cell that overexpresses integrin. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBD10. In some embodiments, the cell-targeting segment is Internalin B or a variant thereof. In some embodiments, the carrier polypeptide is InlBPBD10. In some embodiments, the cell-targeting segment is hepatocyte growth factor (HGF) or a variant thereof. In some embodiments, the carrier polypeptide is HGFPBD10. In some embodiments, the molar ratio of the carrier polypeptide to the positively-charged cytotoxic cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, the method of treating a cancer in a subject comprises administering an effective amount of a composition comprising nanoparticles comprising a carrier polypeptide comprising a penton base segment and a negatively-charged binding segment that binds to a positively-charged cytotoxic polypeptide cargo through an electrostatic interaction. In some embodiments, the carrier polypeptide further comprises a cell-targeting segment. In some embodiments, the carrier polypeptide targets a cancer cell that overexpresses integrin. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBD10. In some embodiments, the cell-targeting segment is Internalin B or a variant thereof. In some embodiments, the carrier polypeptide is InlBPBD10. In some embodiments, the cell-targeting segment is hepatocyte growth factor (HGF) or a variant thereof. In some embodiments, the carrier polypeptide is HGFPBD10. In some embodiments, the cytotoxic polypeptide cargo comprises a protein-synthesis inhibitor, such as gelonin or a variant thereof. In some embodiments, the cytotoxic polypeptide cargo is about 5 kDa to about 50 kDa. In some embodiments, the cytotoxic polypeptide cargo is less than about 5 kDa. In some embodiments, the molar ratio of the carrier polypeptide to the cytotoxic polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

In some embodiments, the method of treating a cancer in a subject comprises administering an effective amount of a composition comprising nanoparticles comprising a carrier polypeptide comprising a penton base segment and a negatively-charged binding segment comprising poly-aspartic acid (such as deca-aspartic acid) or poly-glutamic acid that binds to a positively-charged cytotoxic polypeptide cargo through an electrostatic interaction. In some embodiments, the carrier polypeptide targets a cancer cell that overexpresses integrin. In some embodiments, the carrier polypeptide further comprises a cell-targeting segment. In some embodiments, the cell-targeting segment targets a diseased cell, such as a cancer cell (for example, a HER3+ cancer cell or a c-MET+ cancer cell). In some embodiments, the cell-targeting segment is Heregulin or a variant thereof. In some embodiments, the carrier polypeptide is HerPBD10. In some embodiments, the cell-targeting segment is Internalin B or a variant thereof. In some embodiments, the carrier polypeptide is InlBPBD10. In some embodiments, the cell-targeting segment is hepatocyte growth factor (HGF) or a variant thereof. In some embodiments, the carrier polypeptide is HGFPBD10. In some embodiments, the cytotoxic polypeptide cargo comprises a protein-synthesis inhibitor, such as gelonin or a variant thereof. In some embodiments, the cytotoxic polypeptide cargo is about 5 kDa to about 50 kDa. In some embodiments, the cytotoxic polypeptide cargo is less than about 5 kDa. In some embodiments, the molar ratio of the carrier polypeptide to the cytotoxic polypeptide cargo is about 3:1 to about 8:1. In some embodiments, the average size of the nanoparticles in the composition is about 100 nm or less. In some embodiments, the polydispersity index of the nanoparticles in the composition is about 0.3 or less.

### Articles of Manufacture and Kits

Also provided are articles of manufacture comprising the compositions described herein in suitable packaging. Suitable packaging for compositions described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

The present invention also provides kits comprising compositions (or articles of manufacture) described herein and may further comprise instruction(s) on methods of using the composition, such as uses described herein. The kits described herein may further include other materials desirable from a commercial and user standpoint, including other buffers, diluents, filters, needles, syringes, and package inserts with instructions for performing any methods described herein.

### Exemplary Embodiments

The following embodiments are exemplary and are not intended to limit the scope of the invention or inventions described herein.

Embodiment 1. A composition comprising nanoparticles comprising:

a carrier polypeptide comprising a penton base segment and a binding segment; and

a polypeptide cargo comprising a tag segment that binds to the binding segment of the carrier polypeptide through an electrostatic interaction.

Embodiment 2. The composition of embodiment 1, wherein the tag segment is heterologous to the rest of the polypeptide cargo.

Embodiment 3. The composition of embodiment 1, wherein the tag segment is autologous to the rest of the polypeptide cargo.

Embodiment 4. The composition of any one of embodiments 1-3, wherein the tag segment is at the C-terminus or the N-terminus of the polypeptide cargo.

Embodiment 5. The composition of embodiment 4, wherein the tag segment is cleavable.

Embodiment 6. The composition of any one of embodiments 1-3, wherein the tag segment is internal to the polypeptide cargo.

Embodiment 7. The composition of any one of embodiments 1-6, wherein the tag segment is about 4 amino acids to about 20 amino acids in length.

Embodiment 8. The composition of any one of embodiments 1-7, wherein the binding segment is positively charged.

Embodiment 9. The composition of any one of embodiments 1-8, wherein the binding segment comprises poly-lysine or poly-arginine.

Embodiment 10. The composition of any one of embodiments 1-9, wherein the binding segment comprises decalysine.

Embodiment 11. The composition of any one of embodiments 1-10 wherein the tag segment is negatively charged.

Embodiment 12. The composition of any one of embodiments 1-11, wherein the tag segment comprises poly-aspartic acid or poly-glutamic acid.

Embodiment 13. The composition of any one of embodiments 1-12, wherein the tag segment comprises deca-aspartic acid.

Embodiment 14. The composition of any one of embodiments 1-7, wherein the binding segment is negatively charged.

Embodiment 15. The composition of any one of embodiments 1-7 and 14, wherein the binding segment comprises poly-aspartic acid or poly-glutamic acid.

Embodiment 16. The composition of any one of embodiments 1-7, 14, and 15, wherein the binding segment comprises deca-aspartic acid.

Embodiment 17. The composition of any one of embodiments 1-7 and 14-16, wherein the tag segment is positively charged.

Embodiment 18. The composition of any one of embodiments 1-7 and 14-17, wherein the tag segment comprises poly-lysine or polyarginine.

Embodiment 19. The composition of any one of embodiments 1-7 and 14-18, wherein the tag segment comprises deca-lysine.

Embodiment 20. The composition of any one of embodiments 1-19, wherein the polypeptide cargo comprises a therapeutic polypeptide.

Embodiment 21. The composition of any one of embodiments 1-20, wherein the polypeptide cargo comprises a cytotoxic polypeptide.

Embodiment 22. The composition of embodiment 21, wherein the cytotoxic polypeptide is a protein-synthesis inhibitor.

Embodiment 23. The composition of embodiment 22, wherein the protein-synthesis inhibitor is gelonin or a variant thereof.

Embodiment 24. The composition of any one of embodiments 1-23, wherein the polypeptide cargo is about 5 kDa to about 50 kDa.

Embodiment 25. The composition of any one of embodiments 1-22, wherein the polypeptide cargo is less than about 5 kDa.

Embodiment 26. The composition of any one of embodiments 1-25, wherein the molar ratio of the carrier polypeptide to the polypeptide cargo is about 3:1 to about 8:1.

Embodiment 27. The composition of any one of embodiments 1-26, wherein the carrier polypeptide further comprises a cell-targeting segment.

Embodiment 28. The composition of embodiment 27, wherein the cell-targeting segment binds a mammalian cell.

Embodiment 29. The composition of embodiment 27 or 28, wherein the cell-targeting segment binds a diseased cell.

Embodiment 30. The composition of any one of embodiments 27-29, wherein the cell-targeting segment binds a cancer cell.

Embodiment 31. The composition of embodiment 30, wherein the cancer cell is a HER3+ cancer cell or a c-MET+ cancer cell.

Embodiment 32. The composition of any one of embodiments 27-31, wherein the cell-targeting segment binds a target molecule on the surface of a cell.

Embodiment 33. The composition embodiment 32, wherein the target molecule is a receptor.

Embodiment 34. The composition of embodiment 33, wherein the receptor is HER3 or c-MET.

Embodiment 35. The composition of embodiment 33 or 34, wherein the cell-targeting segment comprises a ligand that specifically binds the receptor.

Embodiment 36. The composition of embodiment 35, wherein the cell-targeting segment comprises:

(i) Heregulin or a variant thereof;

(ii) Internalin B or a variant thereof; or

(iii) hepatocyte growth factor or a variant thereof.

Embodiment 37. The composition of any one of embodiments 1-36, wherein the penton base segment comprises an amino acid sequence according to SEQ ID NO: 1.

Embodiment 38. The composition of any one of embodiments 1-36, wherein the penton base segment comprises a penton base variant.

Embodiment 39. A carrier polypeptide comprising a penton base segment and a negatively-charged binding segment.

Embodiment 40. The carrier polypeptide of embodiment 39, wherein the negatively-charged binding segment comprises poly-aspartic acid.

Embodiment 41. The carrier polypeptide of embodiment 39 or 40, wherein the negatively-charged binding segment comprises deca-aspartic acid.

Embodiment 42. The carrier polypeptide of any one of embodiments 39-41, wherein the carrier polypeptide further comprises a cell-targeting segment.

Embodiment 43. The carrier polypeptide of embodiment 42, wherein the cell-targeting segment binds a mammalian cell.

Embodiment 44. The carrier polypeptide of embodiment 42 or 43, wherein the cell-targeting segment binds a diseased cell.

Embodiment 45. The carrier polypeptide of any one of embodiments 42-44, wherein the cell-targeting segment binds a cancer cell.

Embodiment 46. The carrier polypeptide of embodiment 45, wherein the cancer cell is a HER3+ cancer cell or a c-MET+ cancer cell.

Embodiment 47. The carrier polypeptide of any one of embodiments 42-46, wherein the cell-targeting segment binds a target molecule on the surface of a cell.

Embodiment 48. The carrier polypeptide embodiment 47, wherein the target molecule is a receptor.

Embodiment 49. The carrier polypeptide of embodiment 48, wherein the receptor is HER3 or c-MET.

Embodiment 50. The carrier polypeptide of embodiment 48 or 49, wherein the cell-targeting segment comprises a ligand that specifically binds the receptor.

Embodiment 51. The carrier polypeptide of embodiment 50, wherein the cell-targeting segment comprises:

(i) Heregulin or a variant thereof;

(i) Internalin B or a variant thereof; or

(iii) hepatocyte growth factor or a variant thereof.

Embodiment 52. The carrier polypeptide of any one of embodiments 39-51, wherein the penton base segment comprises an amino acid sequence according to SEQ ID NO: 1.

Embodiment 53. The carrier polypeptide of any one of embodiments 39-52, wherein the penton base segment comprises a penton base variant.

Embodiment 54. A composition comprising nanoparticles comprising:

the carrier polypeptide of any one of embodiments 39-53; and

a positively-charged cargo bound to the negatively-charged binding segment of the carrier polypeptide through an electrostatic interaction.

Embodiment 55. The composition of embodiment 54, wherein the cargo is a polypeptide cargo.

Embodiment 56. The composition of any embodiment 54 or 55, wherein the cargo comprises a therapeutic agent.

Embodiment 57. The composition of any one of embodiments 54-56, wherein the cargo comprises a cytotoxic agent.

Embodiment 58. The composition of any one of embodiments 1-38 and 54-57, wherein the average size of the nanoparticles in the composition is about 100 nm or less.

Embodiment 59. The composition of any one of embodiments 1-38 and 54-58, wherein the nanoparticles in the composition have a polydispersity index of about 0.1 or less.

Embodiment 60. A pharmaceutical composition comprising the composition of any one of embodiments 1-38 and 54-59, further comprising a pharmaceutically acceptable excipient.

Embodiment 61. A method of treating a disease in a subject comprising administering an effective amount of the composition according to any one of embodiments 1-38 and 54-60 to the subject.

Embodiment 62. The method of embodiment 61, wherein the disease is cancer.

Embodiment 63. The method of embodiment 61 or 62, further comprising administering an additional therapy to the subject.

Embodiment 64. The method of embodiment 63, wherein the additional therapy is administered prior to administering the composition comprising the nanoparticles.

Embodiment 65. The method of embodiment 63, wherein the additional therapy is administered after administering the composition comprising the nanoparticles.

Embodiment 66. The method of embodiment 63, wherein the additional therapy is administered contemporaneous to administering the composition comprising the nanoparticles.

Embodiment 67. The method of any one of embodiments 63-66, wherein the additional therapy comprises administering a HER2 antibody to the subject.

Embodiment 68. The method of embodiment 67, wherein the HER2 antibody is trastuzumab, pertuzumab, or a combination thereof.

Embodiment 69. The method of any one of embodiments 63-68, wherein the additional therapy comprises administering a HER2 inhibitor.

Embodiment 70. The method of embodiment 69, wherein the HER2 inhibitor is lapatinib.

Embodiment 71. The method of any one of embodiments 62-70, wherein:

the cancer is a HER3+ cancer and the carrier polypeptide comprises a cell-targeting segment that binds to HER3; or

the cancer is a c-MET+ cancer and the carrier polypeptide comprises a cell-targeting segment that binds to c-MET.

Embodiment 72. A method of making the nanoparticle composition according to any one of embodiments 1-38 and 54-60 comprising combining the carrier polypeptide and the cargo.

Embodiment 73. A method of delivering a polypeptide cargo to a cell comprising contacting the cell with the composition according to any one of embodiment 1-38 and 54-60.

### Examples

The examples provided herein are included for illustrative purposes only and are not intended to limit the scope of the invention.

### Example 1: Nanoparticle Assembly

Nanoparticles comprising a carrier polypeptide and a polypeptide cargo were assembled using the following methods. Green fluorescent protein with a deca-aspartic acid tag segment (GFPD10) (SEQ ID NO: 10) cloned onto the C-terminal end of the green fluorescent protein was incubated with a carrier polypeptide (HerPBK10) comprising a Her cell-targeting segment, a penton base (PB) segment, a decalysine (K10) binding segment, and an N-terminal purification tag (SEQ ID NO: 11) at a molar ratio of 1:3 HerPBK10:GFPD10 in a HEPES-buffered saline. The binding segment of the cargo polypeptide (D10 segment of the GFPD10) binds to the binding segment of the carrier polypeptide (K10), as shown in FIG. 1C. The mixture of carrier polypeptide and GFPD10 was rocked on ice, thereby forming the HerPBK10-GFPD10 particles.

Separately, gelonin with a deca-aspartic acid tag (GeloninD10) cloned onto the C-terminal end of the green fluorescent protein was incubated with a carrier polypeptide (HerPBK10) comprising a Her cell-targeting segment, a penton base (PB) segment, a decalysine (K10) binding segment, and an N-terminal purification tag at a molar ratio of 1:3 HerPBK10:GeloninD10 in a HEPES-buffer in saline. The mixture of the carrier polypeptide and GeloninD10 was rocked on ice, thereby forming the HerPBK10-GeloninD10 particles.

The resulting nanoparticles were then subjected to ultracentrifugation. Specifically, sterile HBS was added to a 50 kD cut-off Centrifugal Filter (Amicon Ultra-15) that had been pre-incubated in sterile, 10% glycerol for 24 hours. The HerPBK10-GFPD10 mixtures were added to the cold HBS in the centrifugal filer. The filter tubes were then spun for 10-20 minutes at 2500 RPM (5000×g) in a Beckman J6-HC centrifuge until the final volume was between 200 μL and 500 μL. The concentrated HerPBK10-GFPD10 or HerPBK10-GeloninD10 was then transferred to a 1.7 mL microfuge tube.

### Example 2: Nanoparticle Size

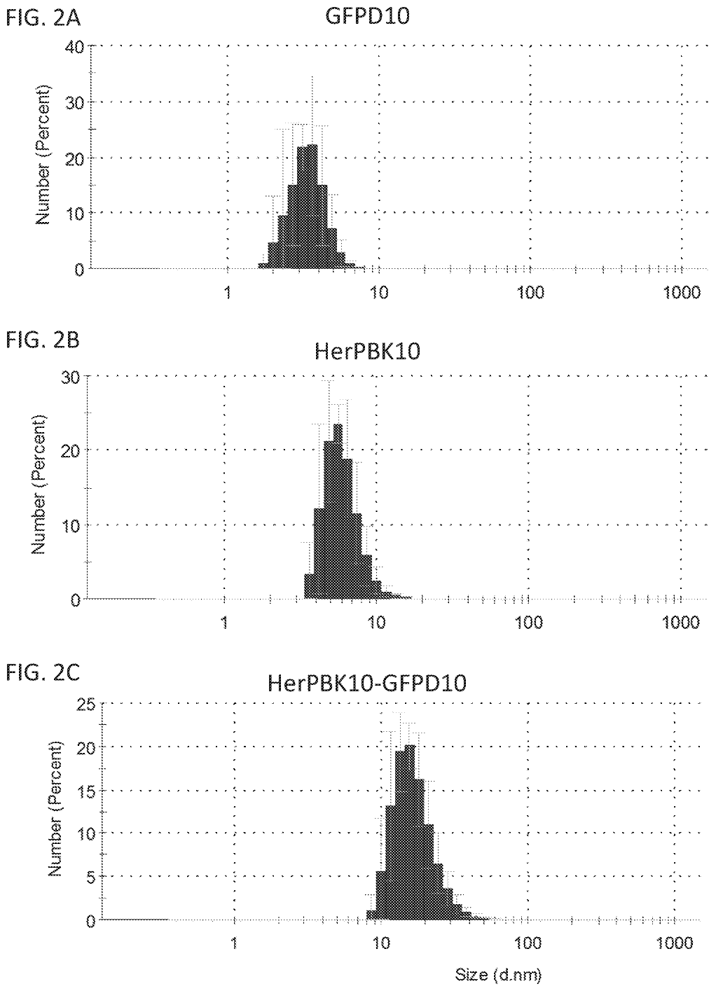

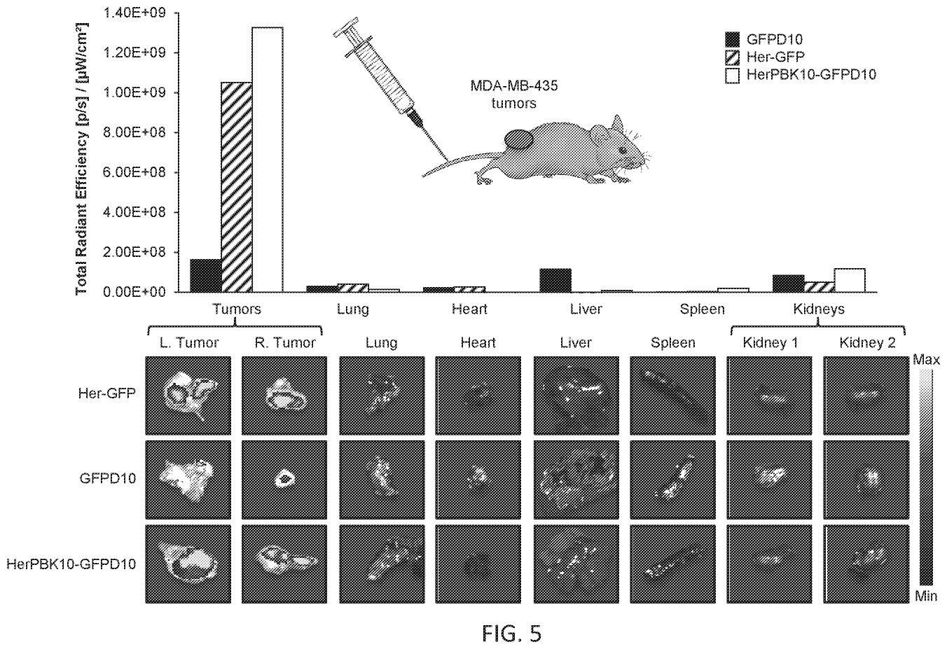

The composition comprising the HerPBK10-GFPD10 nanoparticles and the composition comprising the HerPBK10-GeloninD10 were then subjected to dynamic light scattering (DLS) to determine the diameter of the resulting nanoparticles. Solutions of GFPD10 (no HerPBK10), GeloninD10 (no HerPBK10) and HerPBK10 (no GFPD10 or GeloninD10) were also measured by DLS. Results are presented in FIG. 2A-F. As seen in FIG. 2C, nanoparticles with an average size of about 19 nm formed when HerPBK10 and GFPD10 were combined, compared with solutions of GFPD10 or HerPBK10, which had an average measured size of about 2.3 nm (FIG. 2A) or about 7.2 nm (FIG. 2B), respectively. Nanoparticles formed when HerPBK10 and GeloninD10 were combined were about 26 nm in diameter (FIG. 2F), whereas GeloninD10 or HerPBK10 had a measured size of about 2.9 nm (FIG. 2D) and 6.6 nm (FIG. 2E), respectively.

### Example 3: Electrostatic Interactions Between Carrier Polypeptide and Polypeptide Cargo

Heparin can be used to verify that the GFPD10 and GeloninD10 cargos bound to the HerPBK10 carrier polypeptide by electrostatic interaction. Heparin possess a strong negative charge that disrupts electrostatic bond formation and should prevent anionic attraction between the GeloninD10 and GFPD10 polypeptide cargos and the HerPBK0 carrier polypeptide. GeloninD10 and GFPD10 are combined with heparin before being mixed with the HerPBK10 carrier polypeptide. Particle size is then determined by dynamic light scattering. The presence of two or more populations of measured diameters indicates that heparin prevents nanoparticle formation by disrupting the electrostatic interaction between the polypeptide cargo and the carrier polypeptide.

For further verification, Gelonin (without the D10 tag segment) and GFP (without the D10 tag segment) are combined with the HerPBK10 polypeptide cargo and particle size is determined by dynamic light scattering. The presence of two or more populations of measured diameters indicates that nanoparticles do not form without the D10 tag segment present on the polypeptide cargo. This further indicates that the electrostatic interaction occurs through the D10 tag segment on the polypeptide cargo.

Binding between the carrier polypeptide cargo and polypeptide cargo are also determined using surface plasmon resonance to measure on and off rates. Either the polypeptide cargo or the carrier polypeptide is immobilized on a chip for the surface plasmon resonance study.

### Example 4: Nanoparticle Composition Stability

Compositions comprising HerPBK10-GFPD10 nanoparticles and compositions comprising HerPBK10-GeloninD10 nanoparticles are tested for stability by incubating the complexes at 3° C., 25° C., or 4° C. for up to 1 month. Aliquots of each composition are taken at regular intervals and the average diameter is measured using dynamic light scattering. Nanoparticles that degrade or separate will show two or more populations of different sizes by dynamic light scattering.

Stability of the HerPBK10-GFPD10 nanoparticles and the HerPBK10-GeloninD10 nanoparticles is also measured in serum (either human serum or mouse serum). The HerPBK10-GFPD10 nanoparticles and the HerPBK10-GeloninD10 nanoparticles are combined with HER3+ cells (MDA-MB-435 cells) with or without serum. The cells are pelleted, washed, lysed, and subject to SDS-PAGE and immunoblotting. The presence of HerPBK10 and the polypeptide cargo (either GeloninD10 or GFPD10) indicates that the nanoparticles remain stable in serum and were successful taken up by the HER3+ cells. The presence of only HerPBK10 (that is, without the polypeptide cargo) indicates that the nanoparticles are not stable in serum or were not successfully taken up by the HER3+ cells.

### Example 5: Nanoparticle Polydispersity

Compositions comprising HerPBK10-GFPD10 or HerPBK10-GeloninD10 were subjected to dynamic light scattering to determine average particle size and distribution from three separate samples. Size distributions by number and intensity (about 20 runs per sample) were measured.

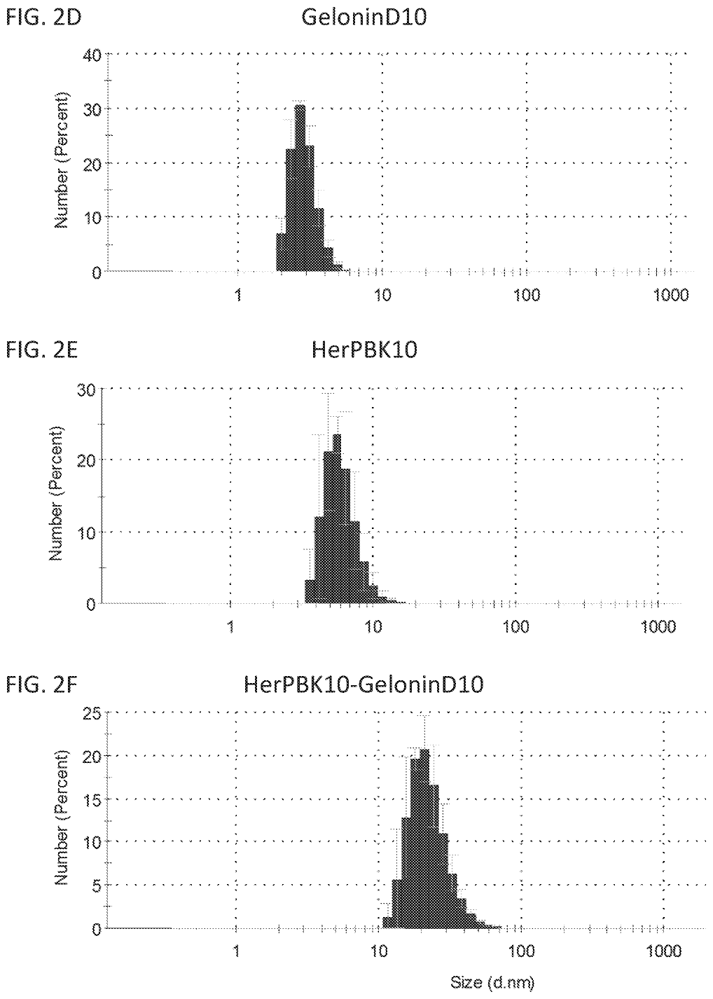

The distribution by number for HerPBK10-GFPD10 nanoparticles are shown in FIG. 3A. The HerPBK10-GFPD10 nanoparticles have a measured average particle size of 17.79 nm with a standard deviation of 0.6868, resulting in a PDI of 0.0014. Accordingly, the HerPBK10-GFPD10 nanoparticles are monodisperse.

The distribution by number for HerPBK10-GeloninD10 nanoparticles are also shown in FIG. 3B. The HerPBK10-GeloninD10 nanoparticles have a measured average particle size of 26.24 nm with a standard deviation of 0.9430, resulting in a PDI of 0.0359. Accordingly, the HerPBK10-GeloninD10 nanoparticles are monodisperse.

### Example 6: Intracellular Trafficking Assay

HerPBK10-GFPD10 nanoparticle delivery was analyzed using a time course intracellular trafficking assay and confocal microscopy. SK-MEL-2 Metastatic Melanoma cells, a cell line that is HER3+, were treated with 20 ng HerPBK10-GFPD10 nanoparticles and stained with DAPI, phalloidin, an anti-AD5 antibody (to recognize the penton base segment on HerPBK10), and a GFP antibody. Images were taken after 0, 15, 30, 60, and 120 minutes using confocal microscopy.

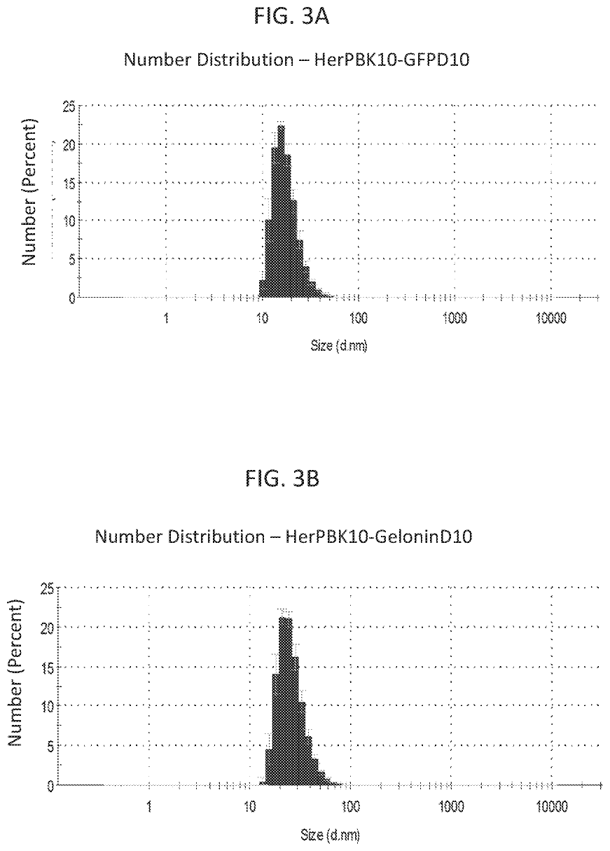

At the 0 minutes time point, HerPBK10 and GFPD10 were colocalized and bound to the cell surface of the HER3+ cancer cell (FIG. 4A). At 15 minutes, HerPBK10 and GFPD10 remained colocalized inside of endosomes (FIG. 4B). At 30 minutes, HerPBK10 escaped the endosome and began to delocalize from GFPD10 (FIG. 4C). At 60 minutes, HerPBK10 and GFPD10 were delocalized and GFPD10 trafficked toward the cytoplasm (FIG. 4D). At 120 minutes, HerPBK10 remained delocalized from GFPD1. GFPD10 had either dissipated and was undetectable or processed within the cell (FIG. 4E). This demonstrates that a carrier polypeptide comprising a cell-targeting segment, a PB cell-penetrating segment, and a cargo binding segment can successfully deliver a polypeptide cargo payload into cells.

### Example 7: Use of Nanoparticles to Kill Cancer Cells

Nanoparticles with carrier polypeptide comprising a cell-targeting segment, a cell-penetrating segment, and a cargo-binding segment and a cargo comprising one or more of various cytotoxic polypeptides, such as a gelonin or other protein-synthesis inhibitors, can be tested for their ability to kill various types of cancer cells.

Various doses of nanoparticle composition can be incubated with either MDA-MB-435 (human cancer) cells, BT474 (human breast cancer) cells, U251 (human glioma) cells, SKOV3 (human ovarian cancer) cells, LNCaP-GFP (human prostate cancer) cells, or RANKL (human bone-metastatic prostate cancer cells).

Relative cell survival after exposure to the described compositions are measured using a cell viability assay. The cells are plated in black-walled, clear-bottom, 96-well plates. 48 hours later, the media is aspirated and replaced with complete media and the indicated concentrations of nanoparticles at a total volume of 40 μL. Plates are rocked for about 4 hours at 37° C. and 5% CO2 and then 60 μL of complete media is added to each well to bring the total volume to 100 μL and the incubation is continued, without rocking, for about 44 hours at 3° C. and 5% CO2. At the conclusion of the incubation, relative cell viability is determined via MTS assay (Promega) according to manufacturer's instructions. Specifically, the media is removed from the wells and 100 μL of fresh complete media is added to each well. 20 μl of the prepared MTS reagent is added to each well. The plate is then incubated with rocking at 37° C. and 5% CO2 and readings are taken of the plate at 1, 2, and 3 hours at 490 nm on spectrophotometer. The results can be shown in terms of the following ratio: number of cells that survived in the treatment group divided by the number of cells that survived in the untreated group. Thus, cell survival of 1.0 indicates that the treated cells and the untreated cells survived to the same extent, whereas a ratio of 0.2 means that as compared with the untreated cell group, only 20% of the treated cells survived.

### Example 8: Carrier Polypeptide with Negatively Charged Cargo-Binding Segment

Nanoparticles comprising a carrier polypeptide with a negatively charged cargo-binding segment and a positively charged cargo are assembled using the following methods.

Positively charged cargo, such as a polypeptide with a positive surface charge, can be incubated with a carrier polypeptide that comprises a negatively charged cargo-binding segment, such as a poly-aspartic acid tail, in HEPES Buffered Saline (HBS). The mixture of carrier polypeptide and cargo can be rocked for 2 hours on ice, thereby forming nanoparticles. Such nanoparticles can be used to traffic cargo into cells.

### Example 9: Biodistribution of GFP in Mouse Xenograft Model

In vivo targeted delivery of green fluorescence protein (GFP) using a HerPBK10 carrier polypeptide was compared to delivery using a fusion polypeptide containing a Her segment and GFP. The Her-GFP fusion polypeptide was prepared as described in Medina-Kauwe et al., Assessing the Binding and the Endocytosis Activity of Cellular Receptors Using GFP-Ligand Fusions, BioTechniques, vol. 29, no. 3, pp. 602-609 (2000). Female nude mice (NU/NU, Charles River) received subcutaneous bilateral flank implants of 1×10⁷ MDA-MB-435 cells per implant. When average tumor sizes reached ˜200 mm³, mice received an injection of HerPBK10-GFPD10, GFPD10 alone, or GFP-HER at 1.5 nmoles GFP per injection. At 6 hours post-injection, the mice were sacrificed and the tissues (including tumors, lung, heart, liver, spleen, and kidneys) were harvested for Xenogen imaging. All tissues were imaged simultaneously with the Xenogen Spectrum imager using 465 nm excitation and 520 nm emission filters. Epifluorescence images were normalized to one another by adjusting the minimum fluorescence values to the same level before calculating average fluorescence intensities of the overall region of each selected tissue.

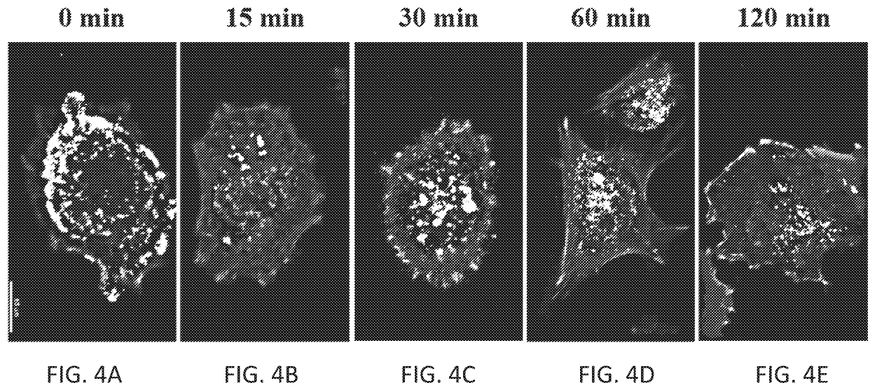

As shown in FIG. 5, GFPD10 was systemically distributed throughout the mouse, with little protein localized to the tumor. Both HerPBK10-GFPD10 particles and Her-GFP fusion polypeptide successfully localized to the MDA-MB-435 tumors. This demonstrates that the HerPBK10 carrier polypeptide can be used to successfully deliver a heterologous polypeptide payload to a tumor without needing to covalently fuse the polypeptide to a targeting ligand.

### Example 10: Intracellular Trafficking of Polypeptide Payloads

The HerPBK10 carrier polypeptide binds to HER3 before inducing endocytosis. This mechanism was explored to see if the HerPBK10 carrier polypeptide could be used to successfully transport a polypeptide carrier into a cell expressing HER3.

HER3 expression levels were measured in MDA-MB-435 cells (HER2+ human breast cancer cells). A375 cells (human malignant melanoma), and A375-MA2 cells. Adherent cells growing in cell culture flasks were washed twice with warm phosphate-buffered saline (PBS); detached with 2 mM EDTA/PBS; collected, washed and pelleted 3-times to remove EDTA; and resuspended in PBS containing fluorescently labeled HER3 antibody (Human ErbB3/HER3 Alexa Fluor 488-conjugated antibody; R&D Systems) at 5 microliters/5×106 cells. Cells were incubated in antibody solution for 30 min with agitation in the dark, followed by gentle pelleting and washing 3-4 times with PBS. After final wash, cell pellets were resuspended in 150 microliters of PBS and analyzed by micro-flow cytometry (Moxi-Go). Cells that were not treated with the antibody were used as a control. Mean fluorescence for each tested cell line is shown in Table 1.

TABLE 1
Mean fluorescence from HER3 antibody

| Cell Line | HER3 | Untreated |
|---|---|---|
| MDA-MB-435 | 65 | 31 |
| A375 | 81 | 28 |
| A375-MA2 | 121 | 28 |

Cells growing on coverslips in 6-well plates were exposed to identical reagents (either HerPBK10 or HerPBK10-GFPD10 particles) at equivalent protein concentrations (20 μg of HerPBK10 per well) according to the procedure established in Rentsendorj et al., Typical and atypical trafficking pathways of Ad5 penton base recombinant protein: implications for gene transfer, Gene Therapy, vol. 13, pp. 821-836 (2006). Cells were fixed after 0, 15, 30, and 60 minutes after exposure to HerPBK10 or HerPBK10-GFPD10 and processed for fluorescence identification of HerPBK10 or HerPBK10-GFPD10. An anti-Ad5 antibody primary antibody was used, which binds the penton base (“PB”) segments of the HerPBK10 polypeptide. Samples were imaged using a Leica SPE laser scanning confocal microscope. Acquired images were imported to Image J software and aplit into individual channels to isolate the green fluorescence (HER3) channel. Individual cells in the green channel were selected and integrated densities measured for each selected area. Results for A375-MA2 cells are shown in FIG. 6 (**** indicates p<0.001; ** indicates p<0.01; N.S, indicates no significance; indicators above each bar show p-value significance relative to the untreated cells).

### Example 11: Cytotoxic Polypeptide Delivery to Cancer Cells

The targeted delivery of the cytotoxic polypeptide gelonin with a C-terminal deca-aspartic acid tag (GeloninD10) using a carrier polypeptide (HerPBK10) was compared to the delivery of GeloninD10 alone using a cell survival assay and an apoptosis assay.

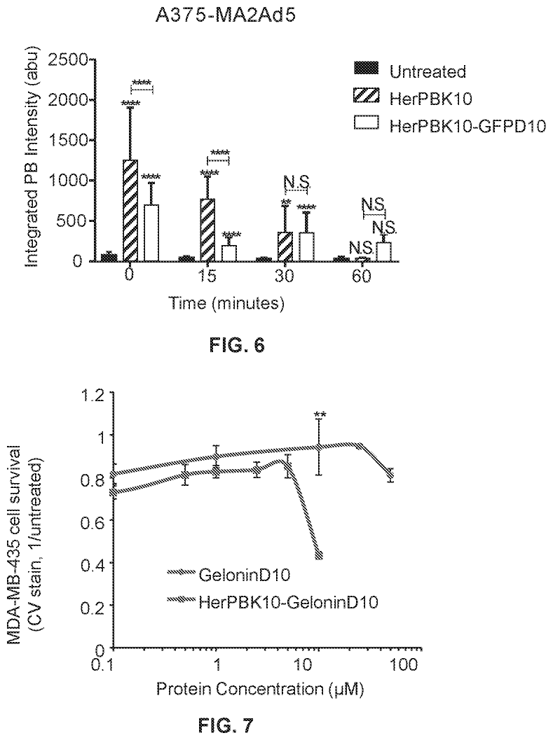

To perform the cell survival assay, sub-confluent MDA-MB-435 cells growing in 96-well dishes were treated with titrating concentrations of HerPBK10-GeloninD10 nanoparticles or GeloninD10. After 24 hours, the cells were fixed and stained using crystal violet to quantify numbers of surviving cells relative to untreated cells, as described in Agadjanian et al., Tumor detection and elimination by a targeted gallium corrole, Proc. Nat'l. Acad. Sci. USA, vol. 106, no. 15, pp. 6105-6010 (2009). Briefly, the cells were washed in phosphate buffered saline before being treated with 0.1% crystal violet in 10% ethanol. The cells were stained for 15 minutes at room temperature before being washed four times with PBS and then with 95% ethanol. Optical density of the samples was detected at 590 nm using a plate reader. Results are shown in FIG. 7 (** indicates a p-value <0.01). Treatment with HerPBK10-GeloninD10 resulted in significant decrease in cell survival compared to treatment with GeloninD10 alone.

An apoptosis assay was performed by treating MDA-MB-435 cells (HER2+ human breast cancer cells with high HER3 expression levels) or MDA-MB-231 cells (HER2− human breast cancer cells and low HER3 expression) were treated with 5 μM HerPBK10-GeloninD10. Reagents for real-time fluorescence measurement of Annexin V binding (indicating apoptosis) were added to the cell cultures, and imaged using an IncuCyte® live cell imager/analyzer (Essen Biosciences) at 0, 12, 24, and 48 hours post-treatment. Significant apoptosis was observed for the MDA-MB-435 cell culture treated with HerPBK10-GeloninD10, with only minimal amounts of apoptosis observed for the untreated MDA-MB-435 cells, or the treated or untreated MD-MB-231 cells.

What is claimed is:

1. A composition comprising nanoparticles comprising:
a carrier polypeptide comprising a penton base segment and a binding segment; and
a polypeptide cargo comprising a tag segment that binds to the binding segment of the carrier polypeptide through an electrostatic interaction.

2. The composition of claim 1, wherein the tag segment is heterologous to the rest of the polypeptide cargo.

3. The composition of claim 1, wherein the tag segment is autologous to the rest of the polypeptide cargo.

4. The composition of any one of claims 1-3, wherein the tag segment is at the C-terminus or the N-terminus of the polypeptide cargo.

5. The composition of claim 4, wherein the tag segment is cleavable.

6. The composition of any one of claims 1-3, wherein the tag segment is internal to the polypeptide cargo.

7. The composition of any one of claims 1-6, wherein the tag segment is about 4 amino acids to about 20 amino acids in length.

8. The composition of any one of claims 1-7, wherein the binding segment is positively charged.

9. The composition of any one of claims 1-8, wherein the binding segment comprises poly-lysine or poly-arginine.

10. The composition of any one of claims 1-9, wherein the binding segment comprises decalysine.

11. The composition of any one of claims 1-10 wherein the tag segment is negatively charged.

12. The composition of any one of claims 1-11, wherein the tag segment comprises poly-aspartic acid or poly-glutamic acid.

13. The composition of any one of claims 1-12, wherein the tag segment comprises deca-aspartic acid.

14. The composition of any one of claims 1-7, wherein the binding segment is negatively charged.

15. The composition of any one of claims 1-7 and 14, wherein the binding segment comprises poly-aspartic acid or poly-glutamic acid.

16. The composition of any one of claims 1-7, 14, and 15, wherein the binding segment comprises deca-aspartic acid.

17. The composition of any one of claims 1-7 and 14-16, wherein the tag segment is positively charged.

18. The composition of any one of claims 1-7 and 14-17, wherein the tag segment comprises poly-lysine or polyarginine.

19. The composition of any one of claims 1-7 and 14-18, wherein the tag segment comprises deca-lysine.

20. The composition of any one of claims 1-19, wherein the polypeptide cargo comprises a therapeutic polypeptide.

21. The composition of any one of claims 1-20, wherein the polypeptide cargo comprises a cytotoxic polypeptide.

22. The composition of claim 21, wherein the cytotoxic polypeptide is a protein-synthesis inhibitor.

23. The composition of claim 22, wherein the protein-synthesis inhibitor is gelonin or a variant thereof.

24. The composition of any one of claims 1-23, wherein the polypeptide cargo is about 5 kDa to about 50 kDa.

25. The composition of any one of claims 1-22, wherein the polypeptide cargo is less than about 5 kDa.

26. The composition of any one of claims 1-25, wherein the molar ratio of the carrier polypeptide to the polypeptide cargo is about 3:1 to about 8:1.

27. The composition of any one of claims 1-26, wherein the carrier polypeptide further comprises a cell-targeting segment.

28. The composition of claim 27, wherein the cell-targeting segment binds a mammalian cell.

29. The composition of claim 27 or 28, wherein the cell-targeting segment binds a diseased cell.

30. The composition of any one of claims 27-29, wherein the cell-targeting segment binds a cancer cell.

31. The composition of claim 30, wherein the cancer cell is a HER3+ cancer cell or a c-MET+ cancer cell.

32. The composition of any one of claims 27-31, wherein the cell-targeting segment binds a target molecule on the surface of a cell.

33. The composition claim 32, wherein the target molecule is a receptor.

34. The composition of claim 33, wherein the receptor is HER3 or c-MET.

35. The composition of claim 33 or 34, wherein the cell-targeting segment comprises a ligand that specifically binds the receptor.

36. The composition of claim 35, wherein the cell-targeting segment comprises:
(i) Heregulin or a variant thereof;
(ii) Internalin B or a variant thereof; or
(iii) hepatocyte growth factor or a variant thereof.

37. The composition of any one of claims 1-36, wherein the penton base segment comprises an amino acid sequence according to SEQ ID NO: 1.

38. The composition of any one of claims 1-36, wherein the penton base segment comprises a penton base variant.

39. A carrier polypeptide comprising a penton base segment and a negatively-charged binding segment.

40. The carrier polypeptide of claim 39, wherein the negatively-charged binding segment comprises poly-aspartic acid.

41. The carrier polypeptide of claim 39 or 40, wherein the negatively-charged binding segment comprises deca-aspartic acid.

42. The carrier polypeptide of any one of claims 39-41, wherein the carrier polypeptide further comprises a cell-targeting segment.

43. The carrier polypeptide of claim 42, wherein the cell-targeting segment binds a mammalian cell.

44. The carrier polypeptide of claim 42 or 43, wherein the cell-targeting segment binds a diseased cell.

45. The carrier polypeptide of any one of claims 42-44, wherein the cell-targeting segment binds a cancer cell.

46. The carrier polypeptide of claim 45, wherein the cancer cell is a HER3+ cancer cell or a c-MET+ cancer cell.

47. The carrier polypeptide of any one of claims 42-46, wherein the cell-targeting segment binds a target molecule on the surface of a cell.

48. The carrier polypeptide claim 47, wherein the target molecule is a receptor.

49. The carrier polypeptide of claim 48, wherein the receptor is HER3 or c-MET.

50. The carrier polypeptide of claim 48 or 49, wherein the cell-targeting segment comprises a ligand that specifically binds the receptor.

51. The carrier polypeptide of claim 50, wherein the cell-targeting segment comprises:
(i) Heregulin or a variant thereof;
(i) Internalin B or a variant thereof; or
(iii) hepatocyte growth factor or a variant thereof.

52. The carrier polypeptide of any one of claims 39-51, wherein the penton base segment comprises an amino acid sequence according to SEQ ID NO: 1.

53. The carrier polypeptide of any one of claims 39-52, wherein the penton base segment comprises a penton base variant.

54. A composition comprising nanoparticles comprising:
the carrier polypeptide of any one of claims 39-53; and
a positively-charged cargo bound to the negatively-charged binding segment of the carrier polypeptide through an electrostatic interaction.

55. The composition of claim 54, wherein the cargo is a polypeptide cargo.

56. The composition of any claim 54 or 55, wherein the cargo comprises a therapeutic agent.

57. The composition of any one of claims 54-56, wherein the cargo comprises a cytotoxic agent.

58. The composition of any one of claims 1-38 and 54-57, wherein the average size of the nanoparticles in the composition is about 100 nm or less.

59. The composition of any one of claims 1-38 and 54-58, wherein the nanoparticles in the composition have a polydispersity index of about 0.1 or less.

60. A pharmaceutical composition comprising the composition of any one of claims 1-38 and 54-59, further comprising a pharmaceutically acceptable excipient.

61. A method of treating a disease in a subject comprising administering an effective amount of the composition according to any one of claims 1-38 and 54-60 to the subject.

62. The method of claim 61, wherein the disease is cancer.

63. The method of claim 61 or 62, further comprising administering an additional therapy to the subject.

64. The method of claim 63, wherein the additional therapy is administered prior to administering the composition comprising the nanoparticles.

65. The method of claim 63, wherein the additional therapy is administered after administering the composition comprising the nanoparticles.

66. The method of claim 63, wherein the additional therapy is administered contemporaneous to administering the composition comprising the nanoparticles.

67. The method of any one of claims 63-66, wherein the additional therapy comprises administering a HER2 antibody to the subject.

68. The method of claim 67, wherein the HER2 antibody is trastuzumab, pertuzumab, or a combination thereof.

69. The method of any one of claims 63-68, wherein the additional therapy comprises administering a HER2 inhibitor.

70. The method of claim 69, wherein the HER2 inhibitor is lapatinib.

71. The method of any one of claims 62-70, wherein:
the cancer is a HER3+ cancer and the carrier polypeptide comprises a cell-targeting segment that binds to HER3; or
the cancer is a c-MET+ cancer and the carrier polypeptide comprises a cell-targeting segment that binds to c-MET.

72. A method of making the nanoparticle composition according to any one of claims 1-38 and 54-60 comprising combining the carrier polypeptide and the cargo.

73. A method of delivering a polypeptide cargo to a cell comprising contacting the cell with the composition according to any one of claim 1-38 and 54-60.


SEQUENCE LISTING (11 sequences)

SEQ ID NO: 1 - protein, 571 aa, Human adenovirus 5
MRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDTEQGGGGAGGSNSSGSGAEENSNAAAAAMQPVEDMNDHAIRGDTFATRAEEKRAEAEAAAEAAAPAAQPEVEKPQKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTF

SEQ ID NO: 2 - protein, 10 aa, Artificial Sequence
  Synthetic Construct
KKKKKKKKKK

SEQ ID NO: 3 - protein, 10 aa, Artificial Sequence
  Synthetic Construct
DDDDDDDDDD

SEQ ID NO: 4 - protein, 215 aa, Artificial Sequence
  Synthetic Construct
ELLPPQLKEMKSQESAAGSKLVLRCETSSEYSSLRFKWFKNGNELNRKNKPQNIKIQKKPGKSELRINKASLADSGEYMCKVISKLGNDSASANIAIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELYGGSGGSRS

SEQ ID NO: 5 - protein, 286 aa, Listeria monocytogenes
ETITVPTPIKQIFSDDAFAETIKDNLKKKSVTDAVTQNELNSIDQIIANNSDIKSVQGIQYLPNVTKLFLNGNKLTDIKPLANLKNLGWLFLDENKVKDLSSLKDLKKLKSLSLEHNGISDINGLVHLPQLESLYLGNNKITDITVLSRLTKLDTLSLEDNQISDIVPLAGLTKLQNLYLSKNHISDLRALAGLKNLDVLELFSQECLNKPINHQSNLVVPNTVKNTDGSLVTPEIISDDGDYEKPNVKWHLPEFTNEVSFIFYQPVTIGKAKARFHGRVTQPLKE

SEQ ID NO: 6 - protein, 796 aa, Artificial Sequence
  Synthetic Construct
ELLPPQLKEMKSQESAAGSKLVLRCETSSEYSSLRFKWFKNGNELNRKNKPQNIKIQKKPGKSELRINKASLADSGEYMCKVISKLGNDSASANIAIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELYGGSGGSRSMRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDTEQGGGGAGGSNSSGSGAEENSNAAAAAMQPVEDMNDHAIRGDTFATRAEEKRAEAEAAAEAAAPAAQPEVEKPQKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTFKKKKKKKKKK

SEQ ID NO: 7 - protein, 796 aa, Artificial Sequence
  Synthetic Construct
ELLPPQLKEMKSQESAAGSKLVLRCETSSEYSSLRFKWFKNGNELNRKNKPQNIKIQKKPGKSELRINKASLADSGEYMCKVISKLGNDSASANIAIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELYGGSGGSRSMRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDTEQGGGGAGGSNSSGSGAEENSNAAAAAMQPVEDMNDHAIRGDTFATRAEEKRAEAEAAAEAAAPAAQPEVEKPQKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTFDDDDDDDDDD

SEQ ID NO: 8 - protein, 874 aa, Artificial Sequence
  Synthetic Construct
ETITVPTPIKQIFSDDAFAETIKDNLKKKSVTDAVTQNELNSIDQIIANNSDIKSVQGIQYLPNVTKLFLNGNKLTDIKPLANLKNLGWLFLDENKVKDLSSLKDLKKLKSLSLEHNGISDINGLVHLPQLESLYLGNNKITDITVLSRLTKLDTLSLEDNQISDIVPLAGLTKLQNLYLSKNHISDLRALAGLKNLDVLELFSQECLNKPINHQSNLVVPNTVKNTDGSLVTPEIISDDGDYEKPNVKWHLPEFTNEVSFIFYQPVTIGKAKARFHGRVTQPLKEGGSGGSRMRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDTEQGGGGAGGSNSSGSGAEENSNAAAAAMQPVEDMNDHAIRGDTFATRAEEKRAEAEAAAEAAAPAAQPEVEKPQKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTFKKKKKKKKKK

SEQ ID NO: 9 - protein, 874 aa, Artificial Sequence
  Synthetic Construct
ETITVPTPIKQIFSDDAFAETIKDNLKKKSVTDAVTQNELNSIDQIIANNSDIKSVQGIQYLPNVTKLFLNGNKLTDIKPLANLKNLGWLFLDENKVKDLSSLKDLKKLKSLSLEHNGISDINGLVHLPQLESLYLGNNKITDITVLSRLTKLDTLSLEDNQISDIVPLAGLTKLQNLYLSKNHISDLRALAGLKNLDVLELFSQECLNKPINHQSNLVVPNTVKNTDGSLVTPEIISDDGDYEKPNVKWHLPEFTNEVSFIFYQPVTIGKAKARFHGRVTQPLKEGGSGGSRMRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDTEQGGGGAGGSNSSGSGAEENSNAAAAAMQPVEDMNDHAIRGDTFATRAEEKRAEAEAAAEAAAPAAQPEVEKPQKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTFDDDDDDDDDD

SEQ ID NO: 10 - protein, 249 aa, Artificial Sequence
  Synthetic Construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKDDDDDDDDDD

SEQ ID NO: 11 - protein, 36 aa, Artificial Sequence
  Synthetic Construct
MRGSHHHHHHGMASMTGGQQMGRDLYDDDDKDRWGS